(19) United States
(12) Patent Application Publication
(10) Pub. No.: US 2009/0156530 A1
(43) Pub. Date: Jun. 18, 2009

(54) Norepinepherine Transporter Mutants and Uses Thereof

(76) Inventors: Randy D. Blakely, Brentwood, TN (US); Uhna Sung, New Haven, CT (US)

Correspondence Address: FULBRIGHT & JAWORSKI L.L.P., 600 CONGRESS AVE., SUITE 2400, AUSTIN, TX, 78701, US

(21) Appl. No.: 12/146,188

(22) Filed: Jun. 25, 2008

Related U.S. Application Data
(60) Provisional application No. 60/948,875, filed on Jul. 10, 2007.

(51) Int. Cl. A61K 31/7088; C07H 21/04; C12N 5/10; C12N 15/63; A01K 67/00; G01N 33/567

(52) U.S. Cl. 514/44; 536/23.5; 435/325; 435/320.1; 800/13; 435/7.21

(57) ABSTRACT

The present invention provides norepinepherine transporter (NET) mutants which display altered phosphorylation at site T30 and altered receptor trafficking. Methods for the use of the NET mutants, e.g., screening of compounds which alter NET trafficking, are also provided. A transgenic animal such as a mouse may comprise a NET mutant of the present invention.

This application claims benefit of priority to U.S. Provisional Application Ser. No. 60/948,875, filed Jul. 10, 2007, the entire contents of which are hereby incorporated by reference.

The government owns rights in the present invention pursuant to grant number MH 073662 (US) and MH 58921 from the National Institutes of Health.

### BACKGROUND OF THE INVENTION

1. Field of the Invention

The present invention relates generally to the fields of molecular biology and drug development. More particularly, it concerns norepinepherine transporter (NET) mutants which display altered phosphorylation and/or trafficking properties.

2. Description of Related Art

Norepinepherine transporters (NETs) are an important pharmacological target and affect multiple disease states. The neurotransmitter norepinephrine (NE) regulates multiple facets of peripheral physiology including heart rate, cardiac output, vascular tone and metabolism, and through its actions in the CNS, modulates autonomic, cognitive, and emotional behaviors (Berridge and Waterhouse, 2003; Eisenhofer, 2001). The major mechanism for NE inactivation in both the PNS and CNS is reuptake of released NE by presynaptically localized NETs (Iversen, 1974). NETs are targets of psychoactive agents including cocaine, amphetamines, the tricyclic antidepressants (e.g. desmethylimipramine, DMI), and the norepinephrine-selective reuptake inhibitors (NSRIs), currently prescribed for the treatment of mood, anxiety and attention-deficit disorders (Gorman and Kent, 1999). Dysfunction of NE clearance and/or altered NET density have been associated with attention-deficit, depression, and suicide (Klimek et al., 1997; Pliszka, 2005; Schildkraut, 1965; Wong et al., 2000), as well as cardiovascular disorders (Bohm et al., 1995; Esler et al., 1981). NETs are members of the Na⁺ and Cl⁻ dependent neurotransmitter transporter gene family (SLC6) that also includes transporters for dopamine, serotonin, proline, glycine and GABA (DAT, SERT, PROT, GLYT, and GAT, respectively) (Gether et al., 2006; Pacholczyk et al., 1991). Mice lacking the NET gene exhibits disrupted cardiovascular function and increased stress reactivity (Keller et al., 2006) as well as altered sensitivity to antidepressants and psychostimulants (Xu et al., 2000). Polymorphisms in the human NET gene have been linked to mood and cardiovascular disorders and response to antidepressants (Hahn and Blakely, 2002).

NET trafficking is physiologically and pharmacologically important but poorly understood. The activity of NET and related transporters are modulated by endogenous pathways or drugs that induce changes in transporter trafficking and/or catalytic activity (Blakely et al., 2005; Gether et al., 2006). Activation of Gq-coupled acetylcholine receptors or protein kinase C (PKC), and hormones such as angiotensin II can rapidly change NE uptake, functional changes linked to altered transporter trafficking (Apparsundaram et al., 1998; Savchenko et al., 2003; Sumners and Raizada, 1986). Although NET surface trafficking appears to be a prominent mode of NET regulation, modulation of NET catalytic activity via p38 MAPK-linked pathways has also been reported (Apparsundaram et al., 2001). In turn, these endogenous regulatory pathways appear to also support the actions of psychotropic drugs. For example, amphetamine influences NET surface trafficking via PKC- and CaMK-dependent linked pathways (Kantor et al., 2001; Kantor et al., 2004).

The mechanism by which NET regulation permits coordination of NE release and reuptake pathways is presently unclear. NET ectodomain antibodies have been recently developed, permitting a demonstration of hormone and depolarization-elicited changes in NET surface expression in noradrenergic neurons (Savchenko et al., 2003). These studies suggest that one or more pathways activated by neuronal depolarization can influence NET surface expression/catalytic function in support of synaptic NE signaling. Ca²⁺ may participate in NET trafficking, but the mechanism for this effect has not been elucidated. Ca²⁺ is a critical second messenger in neurons, with cytosolic dynamics dictated by mobilization of Ca²⁺ from intracellular stores and capacitative Ca²⁺ entry as well as depolarization elicited Ca²⁺ influx (Berridge, 1998). Moreover, Ca²⁺-activated kinases including PKC and Ca²⁺ calmodulin kinases (CaMK) have been suggested to participate in the regulation of NET and related transporters (Gadea et al., 2002; Jayanthi et al., 2000; Kantor et al., 1999; Uchida et al., 1998; Uchikawa et al., 1995; Yura et al., 1996), though the underlying mechanisms of NET regulation are unclear.

Clearly, there exists a need for model systems that can be used to evaluate NET trafficking and develop new compounds capable of altering NET trafficking and/or NET activity.

### SUMMARY OF THE INVENTION

The present invention overcomes limitations in the prior art by providing NET mutants which display altered phosphorylation and/or trafficking. These NET mutants may be used to screen for compounds which regulate NET phosphorylation and/or trafficking. Transgenic animals expressing NET mutants of the present invention are also contemplated.

The present invention provides evidence that neuronal regulation of NET involves Ca²⁺-dependent transporter surface trafficking and requires the activities of CaMKI and CaMKII. Additionally, the inventors have identified a single residue in the NET NH₂ terminus, T30, as required for both Ca²⁺-triggered phosphorylation events and transporter surface trafficking.

An aspect of the present invention relates to an isolated nucleic acid sequence encoding a norepinepherine transporter, wherein the norepinepherine transporter comprises a point mutation at or a deletion of the threonine at position 30 (T30) of the norepinepherine transporter. Apart from the point mutation or deletion, the isolated nucleic acid may encode the norepinepherine transporter of a human, a mouse, a rat, or other animal. The nucleic acid may comprises a point mutation at T30, such as T30A or T30E. The isolated nucleic acid sequence may be further defined as comprising SEQ ID NO:1 or SEQ ID NO:2. The mutation may be T30G, T30V, T30L, T301, T30P, T30D, T30F, T30Y, T30W, T30K, T30R, T30H, T30S, T30C, T30M, T30N, T30Q. The nucleic acid may comprises a deletion of T30 of the norepinepherine transporter encoded by the nucleic acid. The nucleic acid may comprise a deletion of amino acids 29-47 of the norepinepherine transporter. The isolated nucleic acid sequence may be further defined as comprising SEQ ID NO: 4.

Another aspect of the present invention relates to a host cell containing a nucleic acid sequence of the present invention. The cell may be a mammalian cell, such as a human, mouse, rat, monkey, chicken, dog, cat, horse, pig, cow, sheep, goat, or hamster cell. The cell may be a neuronal cell or an insect cell. The host cell may further comprise a vector.

Yet another aspect of the present invention relates to a vector comprising an isolated nucleic acid sequence of the present invention. The vector may comprise the nucleic acid sequence of SEQ ID NO: 1, SEQ ID NO: 2, or SEQ ID NO: 4. The nucleic acid sequence may be operatively linked to a promoter that directs the expression of the nucleic acid in a cell. The promoter may be a norepinepherine transporter promoter. The vector may comprise a viral vector, such as an adenoviral vector, an adeno-associated viral vector, a retroviral vector, a lentiviral vector, a herpes viral vector, polyoma viral vector or hepatitis B viral vector.

Another aspect of the present invention relates to a transgenic non-human animal, wherein the transgenic animal expresses a norepinepherine transporter comprising a point mutation at or a deletion of position T30 of the norepinepherine transporter. The norepinepherine transporter may comprises a point mutation at T30, such as T30A or T30E. The mutation may be T30D, T30G, T30V, T30L, T30J, T30P, T30F, T30Y, T30W, T30K, T30R, T30H, T30S, T30C, T30M, T30N, T30Q. The nucleic acid may comprise a deletion of T30 of the norepinepherine transporter encoded by the nucleic acid; for example, the nucleic acid may comprises a deletion of amino acids 29-47 of the norepinepherine transporter. The animal may be a mouse. In certain embodiments, the mouse is a knock-in mouse.

Yet another aspect of the present invention relates to a method of screening a candidate modulator of the norepinephrine transporter (NET) comprising (a) administering said candidate modulator to a transgenic animal of the present invention, and (b) measuring the effect of said candidate modulator on NET trafficking or NET function. The transgenic animal may be a mouse. NET activity may be measured with a transport assay on a transgenic cell from the transgenic animal. The transport assay may comprise a radioactive norepinepherine uptake assay using synaptoneurosome. The transport assay may comprise and electrophysiologic measurement. The measuring may comprise a behavioral test. The behavioral test may be an anxiety test, such as a depression test, a Porsolt-forced swim test, a tail suspension, a chronic stress paradigm test, a heart rate test, or a cardiac output test. The norepinepherine modulator may increase NET trafficking to the cell surface. The norepinepherine modulator may decrease NET trafficking to the cell surface.

Another aspect of the present invention relates to a method of screening for a candidate substance that alters norepinepherine transporter activity or trafficking comprising: (a) providing a cell or cell extract expressing a norepinepherine transporter of claim 1; (b) exposing the cell or cell extract to a candidate substance; (c) measuring binding of the candidate substance to the norepinepherine transporter in step (a); (d) comparing binding of the candidate substance by the norepinepherine transporter of step (a) to binding of the candidate substance by a wild-type norepinepherine transporter, wherein the ability of the candidate substance to bind to the wild-type norepinepherine transporter, but not the norepinepherine transporter of the present invention, indicates that the candidate substance alters norepinepherine transporter activity trafficking. The trafficking to the cell surface of the wild-type norepinepherine transporter may be increased or decreased by the candidate substance.

In certain embodiments, the cell or cell extract is obtained from a mammalian cell or cell extract. The cell or cell extract may be a neuronal cell or cell extract. The candidate substance may comprise a labeled molecule. The method may further comprise the use of a fluorescent plate reader to provide high-throughput screening of candidate substances. The candidate substance may be an antidepressant, a nucleic acid molecule, an organic small molecule, an inorganic small molecule, or an organo-pharmaceutical. The activity of the candidate substance may depend on threonine at position 30 of the amino acid sequence of the wild-type norepinepherine transporter.

Yet another aspect of the present invention relates to a method for treating a neurologic or psychiatric condition comprising administering to a subject in need thereof, a therapeutically effective amount of a norepinepherine transporter modulator identified by a method of the present invention. The neurologic or psychiatric condition may be a mood disorder, anxiety, dysthymic disorder, unipolar affective disorder, unipolar major depressive disorder, a panic disorder, attention deficit disorder, a cardiovascular disease, an organ specific autonomic dysfunction, a bladder specific autonomic dysfunction, or a gut specific autonomic dysfunction. The administering may be intravenously, intradermally, intramuscularly, precutaneously, subcutaneously, intraarterially, or by aerosol. The subject may be a mammal, such as a human.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. NE transport is Ca²⁺ dependent. FIG. 1A, Ca²⁺ up-regulates NE transport. Left: Ca²⁺ increases NE transport (100% to 268.88+/−19.39%). Synaptosomes in KRH/EGTA were transferred to KRH/EGTA and KRH/Ca²⁺ as described in Materials and Methods prior to the addition of radio-labeled NE for NE transport assay. Data is an average of 4 independent transport assays. Ca²⁺ depletion reduces NE transport (100% to 38.33+/−4.25%) in cortical synaptosomes. Synaptosomes in KRH/Ca²⁺ were replaced with KRH/Ca²⁺ or KRH/EGTA as described in Materials and Methods prior to the addition of radio-labeled NE for transport assay. Data is an average of 6 independent transport assays. FIG. 1B, Ca²⁺ increases Vmax and reduces Km of NE transport activity in cortical synaptosomes. Cortical synaptosomes in KRH/EGTA were divided into KRH/EGTA and KRH/Ca²⁺, and incubated at 37° C. for 5 min prior to NE transport assay. Ca²⁺ increased Vmax by 155% from 0.129+/−0.0129 pmol/mg proteins/min (KRH/EGTA) to 0.200+/0.0272 pmol/mg proteins/min (KRH/Ca²⁺). Right: Ca²⁺ decreased Km from 0.29+−0.085 nM (KRH/EGTA) to 0.128+/−0.04 nM (KRH/Ca²⁺). Data are averages of Vmax and Km from 4 independent kinetics assays.

FIGS. 2A-E. Influences of PKC and CaMK on Ca²⁺ modulation of NE transport in cortical synaptosomes. Synaptosomes were incubated with vehicle, 0.1-1 μM BIM, 5 μM KN93, or 10 μM W7 at in KRH/Ca²⁺ for 15 min at 37° C. FIG. 2A, NE transport assays for the vehicle or drug-pretreated synaptosomes were carried out in KRH/Ca²⁺. BIM, KN93 and W7 inhibit NE transport. Data is an average of 6 independent experiments. FIG. 2B, Synaptosomes were pre-incubated with vehicle or drugs in KRH/Ca²⁺ as described below, and washed with KRH/EGTA. Then, the synaptosomes in KRH/EGTA were divided into 2 groups and re-suspended in KRH/EGTA or KRH/Ca²⁺ as described in Example 1 prior to NE transport assay. Data is an average of 6 independent experiments. KN93 inhibited Ca²⁺ induced increase of NE transport whereas BIM lacks effects. FIG. 2C, Synaptosomes pre-incubated with vehicle or drugs in KRH/Ca²⁺ were replaced with fresh KRH/Ca²⁺ or KRH/EGTA as described below prior to NE transport assay. Data is an average of 6 independent experiments. KN93 attenuates Ca²⁺ depletion-induced down-regulation of NE transport whereas BIM lacks effects.

FIGS. 3A-C. Ca²⁺ dependent surface trafficking of NET. CHO cells were transiently transfected with HA-NET. Surface NET was detected by surface biotinylation followed by immunoblotting with anti-HA. FIG. 3A, Ca²⁺ influx in Ca²⁺-depleted CHO cells upon change of Ca²⁺ concentration in external medium. Data represents averaged traces of the response of recordings from 27 individual cells. Standard error bars have been omitted for the clarity but represented no more than 5% of the normalized ratio values. FIG. 3B, Cells were incubated with vehicle or 5 μM KN93 in complete media for 30 min at 37°. Left: Ca²⁺ increased surface NET. After depletion of Ca²⁺ as described in Example 1, cells were replaced with KRH/EGTA or KRH/Ca²⁺ and incubated at RT for 1 min prior to biotinylation. KN93 blocked Ca²⁺ triggered increase of surface NET. A representative immunoblot of surface NET is shown. Band densities are averages from 3 independent experiments. Right: Ca²⁺ depletion diminished NET at the surface. Cells were replaced with complete media containing either vehicle or 10 mM EGTA/30 μM BAPTA/AM and incubated for 10 min prior to surface biotinylation. A representative immunoblot of surface NET is shown. Band densities are averages from 5 independent experiments. Cells pre-incubated with KN93 did not reduce surface NET. FIG. 3C, Inhibition of CaMKK by STO609 inhibits Ca²⁺ dependent surface trafficking in CHO cells. CHO cells were incubated in complete media with 5 μM STO-609 for 1 hour at 37° C. Restoration of Ca²⁺ or depletion of Ca²⁺ is performed as described above. A representative surface biotinylation is shown.

FIGS. 4A-D. CaMKI and CaMKII are responsible for Ca²⁺ dependent surface trafficking of NET. CAD-NET cells were mock-transfected (control) or transiently transfected with siRNAs of CaMKI or CaMKIIδ. FIG. 4A, siRNAs reduced expression of CaMKI and CaMKII, but did not influence protein expression of NET. NETs in total lysates show expression of 60 kDa immature and 90 kDa mature forms. FIG. 4B. Cells were incubated in KRH/Ca²⁺ with vehicle or 5 μM KN93 at 37° C. for 20 min prior to NE transport assay in the same buffer. siRNAs inhibit NE transport. FIG. 4C, siRNAs of CaMKI and CaMKII inhibit Ca²⁺-dependent surface trafficking of NET. Cells were incubated in complete media without or with 10 mM EGTA for 5 min at 37° C. prior to surface biotinylation and immunoblotting with anti-HA. A representative immunoblot of surface NET from 3 independent experiments is shown. The longer-exposed immunoblot for CaMKII siRNA is also shown. FIG. 4D, Average surface band density of 3 independent experiments for FIG. 4C.

FIGS. 5A-E. N-terminal domain of NET is responsible for Ca²⁺-dependent surface trafficking of NET. FIG. 5A, N-terminal cytoplasmic domains of NET (human, rat, and mouse), human DAT, GAT1, and SERT. Alignment was performed using DNASTAR MegAlign 4.0.3 by clustral method. Conserved amino acids are shown in box. The sequence between 28 to 47 amino acids in hNET is marked by a line. hNET contains 3 threonines in NH₂ domain, as indicated by asterisk. FIG. 5B, FIG. 5C, FIG. 5D, CHO cells were transiently transfected with HA-tagged NET, NETΔ28-47 or NET T30A. Manipulation of external Ca²⁺ (increase of Ca²⁺ and depletion of Ca²⁺) was performed as described in Example 1. Surface NET or NET mutants was detected by surface biotinylation followed by immunoblotting with anti-HA. A representative immunoblot of surface NET or NET mutants is shown from each experiment. Band densities are averages from 3-5 independent experiments. FIG. 5B, Ca²⁺ dependent surface trafficking of NET. Left: Ca²⁺ increased NET at the surface within a min and sustained up to 5 min. Right: Ca²⁺ depletion reduced NET from the surface within a min and sustained up to 5 min. FIG. 5C, NETΔ28-47 did not respond to change of external Ca²⁺. Left: Ca²⁺ did not increase surface expressions of NETΔ28-47 up to 5 min. Right: Depletion of Ca²⁺ did not reduce surface number of NETΔ28-47 up to 5 min. FIG. 5D, NET T30A did not respond to change of external Ca²⁺. Left: Ca²⁺ did not increase surface expressions of NET T30A. Right: Depletion of Ca²⁺ did not reduce surface number of NET T30A. FIG. 5E, NE transport activities of NET, NETA28-47 and NET T30A in transiently transfected CHO cells. Left: NE transport activities of NET, NETΔ 28-47 and NET T30A in KRH/Ca²⁺ are similar each other. Data is an average of 4 independent experiments. Right: Recovery of NE transport in KRH/Ca²⁺ after Ca²⁺ depletion. After CHO cells were incubated in KRH/EGTA with 1 μM thapsigargin for 10 min as described in Example 1, cells were replaced with fresh KRH/Ca²⁺, incubated for 5 min, and assayed for NE transport assay in KRH/Ca²⁺ for 10 min. NE transport activities of NET Δ 28-47 and NET T30A after Ca²⁺ depletion were not fully recovered as wild-type NET. Data is an average of 3 independent experiments.

FIGS. 6A-B. Ca²⁺ phosphorylates NET in a T30 dependent manner. FIG. 6A, NET T30E, a mutant mimicking constitutively phosphorylated NET at T30, did not respond to Ca²⁺. CHO cells were transiently transfected with HA-tagged NET T30E. Manipulation of external Ca²⁺ (increase of Ca²⁺ or depletion of Ca²⁺) was performed as described in Materials and Methods. Surface NET or NET T30E was detected by surface biotinylation followed by immunoblot with anti-HA. Left: while NET increase surface number responding to Ca²⁺ within a min, NET T30E did not change surface number. Right: While NET reduced surface number upon depletion of Ca²⁺ in external media, NET T30E did not reduce surface number within 5 min. A representative immunoblot of surface NET is shown. FIG. 6B, Ca²⁺ phosphorylates NET, but does not NET T30A. CHO-NET and CHO-NET T30A cells was phosphorylated and pre-incubated in Ca²⁺ free buffer as described in Materials and Methods. One set of cells were supplemented with 2.2 mM CaCl₂ for 5 min. Immunoprecipitated NET and NET T30A were analyzed by two parallel gels for immunoblotting with anti-NET (left panel) or image processing in phosphoimager (right panel). Ca²⁺ phosphorylated NET and induced an interaction with high molecular weight proteins. One of the representative data from 3 experiments is shown.

FIGS. 7A-G. Depolarization increases surface NET in SCG culture in Ca²⁺ and CaMK dependent manners.

FIGS. 8A-C. NET-mediated currents are enhanced by electrical stimulation of noradrenergic neurons. FIG. 8A, (top) voltage-step protocol used for whole cell patch clamp recordings of NET currents from single SCG neurons. Panel A (bottom left) demonstrates presence of NET-mediated current as defined by incubations in the presence or absence of 5 μM desipramine (DMI) (I_{basal}). Panel A (bottom right) reveals an increase in DMI-sensitive NET currents after a 2 sec depolarizing voltage step at −10 mV prior to the test pulse. FIG. 8B, Time-dependence of the increase in NET currents following return to −50 mV holding potential, prior to the −120 mV test pulse. Data are expressed as the mean ratio ±SEM of the induced NET-mediated current after DMI subtraction and normalization for NET current elicited prior to the 50 mV depolarizing pulse (I_{basal}) (n=3). FIG. 8C, Impact of Ca²⁺ channels and CaMKs on stimulation of NET-mediated currents. NET currents were elicited as described in Panel A in control conditions, in the presence of 200 μM CdCl₂, or after 15 minutes preincubation with KN93 or 20 minutes preincubation with STO609. Data are normalized to the current recorded in control conditions before prepulse stimulation (I_{basal}) and expressed as mean±SEM (n=3 for each condition). The effect of prepulse depolarization is then compared for each condition with the stimulation elicited under control conditions by paired Student's t test; *=P<0.05 and #=P<0.01.

### DESCRIPTION OF ILLUSTRATIVE EMBODIMENTS

The present invention overcomes limitations in the prior art by providing NET mutants (e.g., T30A, T30E, etc.) which display altered phosphorylation and/or trafficking. These NET mutants may be used to screen for compounds which regulate NET phosphorylation and/or trafficking. Transgenic animals expressing NET mutants of the present invention are also contemplated.

The present invention provides the identification of specific sites in NET (e.g., T30) which are critical for NET trafficking. Data in the below examples establish that Ca²⁺ influx and depolarization support enhanced surface trafficking of NET, elevating NE uptake capacity. Ca²⁺-modulated NET trafficking is completely reversible and requires the activity of the Ca²⁺/calmodulin-dependent protein kinases CaMKI and CaMKII. Mutation studies below identify T30 in the NET NH₂ terminus as essential both for Ca²⁺-triggered NET phosphorylation as well as Ca²⁺-dependent NET trafficking. These findings identify a mechanism through which neuronal Ca²⁺ dynamics modulate presynaptic NE uptake capacity and may link receptor and depolarization-elicited NE release with NE inactivation.

### I. T30 MODULATES SURFACE TRAFFICKING OF NET

Calcium (Ca²⁺) is involved with NET trafficking. Trafficking refers to the degree to which receptors are localized to the membrane surface of a cell, and is very regulated in the case of NET. Neurotransmitter uptake via plasma membrane transporters is highly regulated, with catalytic function and/or transporter trafficking sensitive to G-protein and tyrosine kinase-coupled receptor activation (Blakely et al., 2005; Gether et al., 2006). With respect to NET, Gq coupled muscarinic receptors and insulin receptors have been found to effect trafficking-dependent and independent modes of regulation (Apparsundaram et al., 1998; Apparsundaram et al., 2001). How this regulation integrates with the demands imposed by Ca²⁺-dependent vesicular NE release is largely unexplored, although it was recently established that both peripheral and central NE neurons demonstrate depolarization-triggered elevations in surface NET (Savchenko et al., 2003). Interestingly, prior studies have shown that functional activities of multiple SLC6 family transporters, including NET, are sensitive to Ca²⁺ changes in the external media or treatment of cells that disrupt intracellular Ca²⁺ concentrations (Jayanthi et al., 2000; Uchida et al., 1998; Uchikawa et al., 1995). Ca²⁺ is also critical for the receptor-mediated regulation of NET, DAT and SERT (Apparsundaram et al., 1998; Apparsundaram et al., 2001; Zhu et al., 2005). Ca²⁺-activated kinases, including PKCs and CaMKs, Ser/Thr protein phosphatases including PP2A, and transporter-associated associated proteins such as syntaxin 1A have been implicated in Ca²⁺ regulation of monoamine transporters (Blakely et al., 2005).

The inventors have evaluated how changes in intracellular Ca²⁺ support constitutive transporter function as well as the transporter's response to depolarizing conditions. It was found that Ca²⁺ sustains constitutive NE transport through surface trafficking of NET proteins mediated by both CaMKI and CaMKII. Responses to external Ca²⁺ manipulations (or to voltage-elicited Ca²⁺ influx) are rapid, with changes in surface density or NET function evident in seconds to minutes. Bidirectional surface trafficking responses to either Ca²⁺ addition or Ca²⁺ removal depend critically on residue T30 located in the transporter NH₂ terminus, a site also required for Ca²⁺ elicited transporter phosphorylation. Parallel changes in NE surface expression and NET function can be seen in neurons in response to depolarization-elicited Ca²⁺ influx, thus constitutive Ca²⁺/CaMK dependent trafficking mechanisms may be engaged to link NET surface expression to demands imposed by changes in noradrenergic neuron excitability and NE release.

A. Surface Trafficking Supports Ca²⁺ Regulation of NET

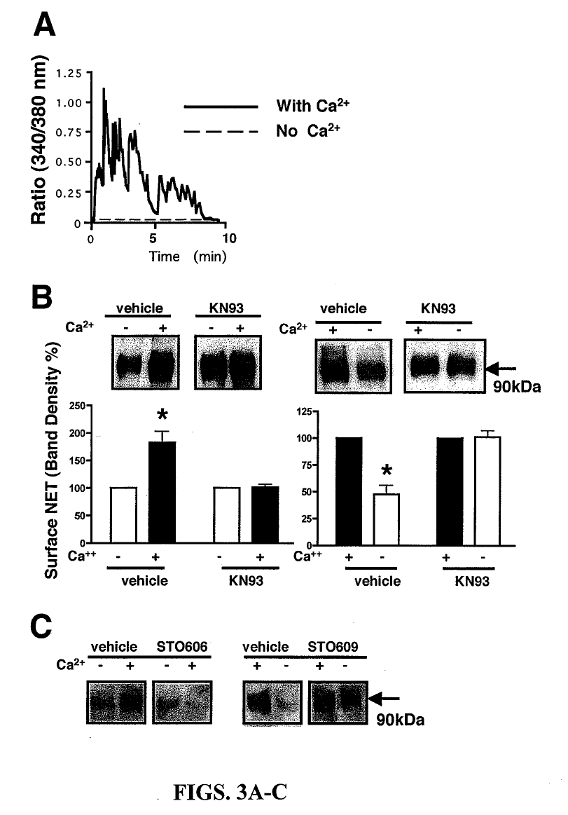

As observed in the below Examples, external medium Ca²⁺ manipulations can bidirectionally alter basal NE transport in cortical synaptosomes. Kinetic exploration of these findings reveal changes in both NET Vmax and Km. Due to the limited sensitivity of NET antibodies in synaptosomes, further exploration of the basis of these observations required monitoring NET in transfected cell models. In established cells, a change in Ca²⁺ concentration in the medium triggers rapid Ca²⁺ mobilization across the plasma membrane (FIG. 3A). As in synaptosomes, medium Ca²⁺ manipulations in normeuronal CHO and neuronal CAD cells bidirectionally alters NE uptake, with steady-state biotinylation studies demonstrating parallel changes in NET surface protein levels. In cultured SCG cells, a NET surface epitope evaluation paradigm was implemented as previously developed (Savchenko et al., 2003) to generate evidence for Ca²⁺ dependent changes in surface NET proteins elicited by depolarization. Together, these findings support transporter trafficking as a key element in relating changes in intracellular Ca²⁺ to transport activity. These observations in synaptosomes of a reduced NE Km suggests that, in parallel with alterations in NET surface density, Ca²⁺ changes also elicit changes in substrate recognition or permeation. A difference was also observed between trafficking and transport modulation when examining loss and recovery of NET activity suggesting additional modulation of function for surface NETs supported by T30. Although speculative at present, such changes may engage p38 MAPK as Ca²⁺ dependent activation of this pathway has previously been demonstrated to alter NET and SERT catalytic rates (Apparsundaram et al., 2001; Zhu et al., 2005).

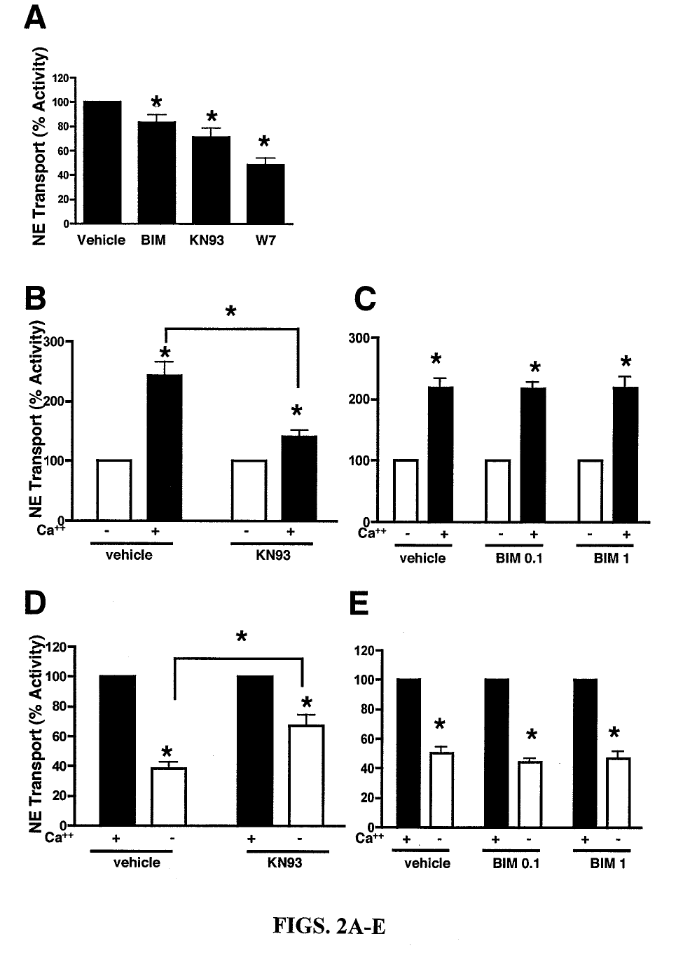

To evaluate the signaling pathways supporting Ca²⁺-dependent NET surface trafficking, the inventors focused on PKC and CaMK pathways as these kinases have been shown to regulate activities of NET and other monoamine transporters (Gadea et al., 2002; Jayanthi et al., 2000; Kantor et al., 1999; Uchida et al., 1998; Uchikawa et al., 1995; Yura et al., 1996). Using BIM and KN93 to inhibit PKCs and CaMKs, respectively, the inventors found that CaMKs are important for Ca²⁺-dependent regulation of NET whereas PKCs participate little in this regulation (FIGS. 2A-E). Syntaxin 1A is known to dictate NET surface trafficking and also to interact with NET to modulate transporter function (Sung et al., 2003). Without wishing to be bound by any theory, the effects observed appear to only indirectly engage syntaxin 1A as engaged in the fusion of NET vesicles. Thus, whereas botulinum toxin C cleavage of syntaxin 1A inhibits NET activity in Ca²⁺ containing, but not Ca²⁺ free, conditions, direct syntaxin 1A/NET associations are sensitive to PKC activating phorbol esters but insensitive to KN93. Attribution of KN93 effects as related to CaMK inhibition is supported by findings with siRNAs for CaMKI and CaMKII. In normal Ca²⁺ medium, KN93 acts to reduce NE uptake activity and NET surface trafficking, suggesting that constitutively active CaMKs provide for enhanced transporter surface expression under basal conditions. This surmise is supported by the findings that Ca²⁺ depletion reduces NET surface expression and that siRNA reduction attenuates or abolishes the effects of Ca²⁺ depletion and of KN93.

B. Coordinated Action of CaMKI and CaMKII in Ca²⁺-Dependent NET Surface Trafficking

CaMKI and CaMKII are involved in Ca2+-dependent NET surface trafficking. KN93 is known to inhibit both CaMKI and CaMKII in vitro with almost identical Kᵢ (Hook and Means, 2001). Experiments using RNA interference and pharmacological inhibition of CaMKK (and consequent inhibition of CaMKI) by STO-609 reveal distinct but overlapping roles of CaMK isoforms in Ca²⁺ dependent NET surface trafficking.

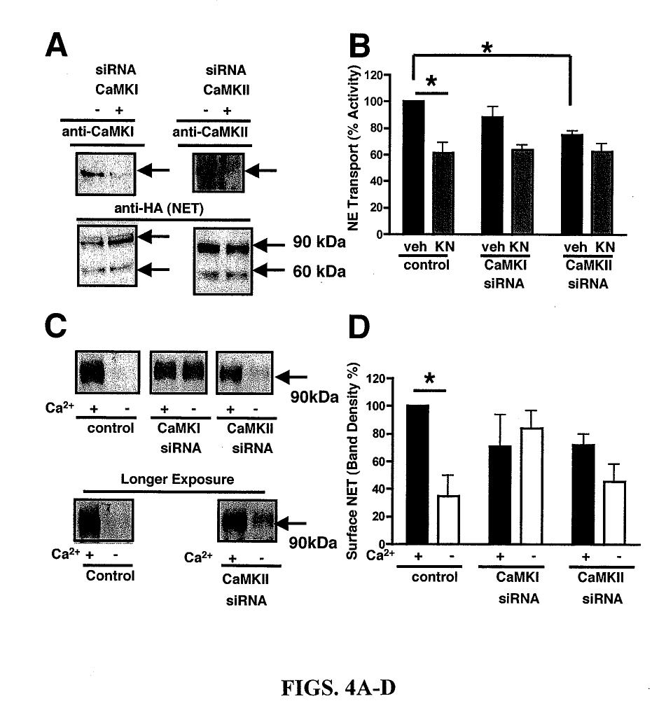

CaMKII appears to regulate NET at two different levels. CaMKII regulates NET under basal conditions, as shown by findings that CaMKII siRNA inhibits basal NE transport to a greater degree than siRNA targeted to CaMKI (FIG. 4B) despite equivalent reductions in the respective kinase. Protein levels or variations in isoform expression do not take into account catalytic rate or localization of each kinase. However, with Ca²⁺ depletion experiments, siRNAs reveal a greater role of CaMKI in NET surface trafficking. In fact, STO-609 did not influence basal NE transport and additional KN93 was still able to suppress NE transport, further supporting marginal roles of CaMKI in basal NE transport in CAD cells. These findings suggest that both CaMKI and CaMKII likely collaborate in the Ca²⁺ dependent trafficking regulation of NET, though their roles may be different. The findings with STO-609 inhibition of depolarization-elicited NET currents in SCG neurons suggest the possibility that CaMKII may regulate constitutive surface trafficking of NET whereas CaMKI may contribute further at the events of acute mobilization of Ca²⁺.

In addition to the effects observed herein, CaMKI an CaMKII have many other functions in the brain. Whereas ample evidence is available for the presynaptic and postsynaptic roles and targets of CaMKII in synaptic plasticity (Xia and Storm, 2005), physiological roles of CaMKI are less understood. CaMK I is expressed widely with high expression in brain including frontal cortex, hippocampus, and locus coeruleus (Picciotto et al., 1993; Rina et al., 2001). CaMKI can be activated by depolarization in PC12 cells and in primary hippocampal neurons (Aletta et al., 1996; Uezu et al., 2002), demonstrates increased expression upon induction of LTP, is important for NMDA-receptor mediated Ca²⁺ elevation with ERK-dependent LTP, and influences activity dependent dendritic development (Schmitt et al., 2005; Tokuda et al., 1997; Wayman et al., 2006). Presynaptic roles of CaMKI have been suggested derived from its ability to phosphorylate synapsin I and from the reports that it regulates growth cone mobility and axonal growth (Picciotto et al., 1993; Wayman et al., 2004). CaMKI phosphorylation of synapsin 1 supports mobilization of synaptic vesicles (Chi et al., 2003). A more general role in the mobilization of NET containing vesicles by CaMKI and CaMKII would be consistent with the changes in NET trafficking observed.

C. T30 Dictates Ca²⁺-Dependent NET Phosphorylation and Surface Trafficking

The NH₂-terminal domain of NET appears to be responsible for Ca²⁺-dependent trafficking modulation. Deletion of NET at residues 28-47 or substitutions of residue T30 results in expression at wildtype levels, maturation and translocation of transporters to the surface, with rates of NE transport as wildtype. However, these mutants lack the ability to respond to Ca²⁺ either with respect to Ca²⁺ depletion or Ca²⁺ restoration to depleted medium. Interestingly, the sequence between 28-47 of NET is divergent from the corresponding NH₂-terminal regions of SERT and DAT although T30 is conserved in mammalian NETs, suggesting that this site may support a NET-specific mechanism to link transport changes to levels of intracellular Ca²⁺. In metabolic labeling studies, evidence is provided below that NET and a NET-associated protein are targets of phosphorylation following Ca²⁺ supplementation. As the T30A mutant that blocks trafficking effects linked to Ca²⁺ manipulations also blocks recovery of both phosphorylated NET T30A and associated phosphoproteins, T30-dependent phosphorylation and NET trafficking appear to be linked. Without wishing to be bound by any theory, NET may become phosphorylated following Ca²⁺ elevations and recruit another phosphoprotein that in turn impacts NET trafficking. Studies using comparative proteomic approaches may allow us to identify the NET-associated phosphoprotein and add further depth to this mechanism. Interestingly, phosphorylation of NET at T258/S259 has recently been reported in association with Ca²⁺-independent, PKCε-linked internalization of NET (Jayanthi et al., 2006) with no effect on transporter insertion or recycling. In contrast, it was found that T30-dependent phosphorylation correlates with Ca²⁺-dependent increases in NET surface density, possibly arising from elevated transporter insertion rates. Using two ectodomain antibodies derived from different species, but targeted to the same epitope, evidence was obtained that depolarization-elicited NET surface elevations arise from directed insertion. These findings suggest that multiple phosphorylation events modulate NET surface trafficking, though likely at different stages in the trafficking cycle.

The NET NH₂ terminal domain is known to be important for the interaction with other cellular proteins. The SNARE protein syntaxin 1A interacts with resides 2-42 (Sung et al., 2003) and Ca²⁺ can alter the interactions of these two proteins (Sung and Blakely, submitted). However, in the latter study KN93 inhibits the Ca²⁺-dependent changes in NET surface trafficking without affecting NET/syntaxin interactions. The NH₂-terminus of NET as also interacts with 14-3-3 proteins (Sung et al., 2005) and PP2A (Sung et al, 2005). Interestingly, CaMKII constitutively interacts with the DAT COOH terminus, and more weakly with the NET COOH terminus, to regulate DA efflux of DAT, a process involved with phosphorylation of residues in the DAT NH₂ terminus. Recently, immunoprecipitations were analyzed from NET transfected CAD cells by LC-MS/MS (liquid chromatography coupled tandem mass spectrometry) (Sung et al., 2005). In these analyses, spectra were obtained matching multiple peptides of CaMKI (FTCEQALQHPWIAGDTALDK (SEQ ID NO:5), NIHQSVSEQIK (SEQ ID NO:6), YLHDLGIVHR (SEQ ID NO:7), XCorr=3.0, 2.4, 2.3, respectively, the XCorr is the cross correlation value for MS analysis), CaMKIIδ (ICDPGLTAFEPEALGNLVEGMDFHR (SEQ ID NO:8), XCorr=4.9), and calmodulin (EAFSLFDKDGDGTITTK (SEQ ID NO:9), EADIDGDGQVNYEEFVQMMTAK (SEQ ID NO:10), SLGQNPTEAELQDMI (SEQ ID NO:11)-NEVDADGNGTIDFPEFLTM (SEQ ID NO:12), DGNGYISAAELR (SEQ ID NO:13), XCorr-3.6, 3.5 and 3.1, 2.9, 1.9, respectively) that were absent from NET-complexes when immunoprecipitations were conducted with parental CAD cells-expressing CAD cells, but one (CaMKI) and none (CaMKII) were recovered from parallel immunoprecipitations conducted with parental CAD cells. Five spectra were found matching to 4 peptides of calmodulin (EAFSLFDKDGDGTITTK (SEQ ID NO:9), EADIDGDGQVNYEEFVQMMTAK (SEQ ID NO:10), SLGQNPTEAELQDMI (SEQ ID NO:11)-NEVDADGNGTIDFPEFLTM (SEQ ID NO:12), DGNGYISAAELR (SEQ ID NO:13), XCorr=3.6, 3.5/3.1, 2.9, 1.9, respectively) from NET-expressing CAD cells versus one from parental CAD cells. Given the evidence of CaMKIV/PP2A complex (Westphal et al., 1998), whether CaMKI or CaMKII as it seems possible that NET assembles with a a signaling complex including one or more CamKs and PP2A in support of NET trafficking.

D. Ca²⁺ in Support of Pre-Synaptic NET Activity

As shown in the below Examples, Ca²⁺ regulates NET surface expression through CaMKI and CaMKII. Subsequent to studies in a heterologous model, the inventors demonstrated that Ca²⁺ is a critical mediator of both basal and depolarization-triggered NET trafficking in transformed and primary neuronal cultures. These findings suggest that CaMKI and CaMKII-dependent trafficking processes establish quantitatively appropriate levels of NE uptake at rest and under conditions of neuronal excitation. As Ca²⁺ is an essential second messenger for excitation-coupled vesicular NE release, release and uptake of NE may be likely tightly coordinated through the actions of CaMKI and CaMKII to effect the fusion of NE vesicles in parallel with the fusion of NET vesicles (Nichols et al., 1990; Schweitzer et al., 1995). Interestingly, evidence has been put forward that NETs may actually reside on NE secretory vesicles derived from adrenal gland and PC12 cells (Kippenberger et al., 1999), though this idea remains to be tested in neurons. It is possible that alterations in CaMK signaling can account for the recent findings that NET surface trafficking is altered in noradrenergic neurons in animals subjected to chronic stress paradigms that triggers elevations in noradrenergic excitation (Miner et al., 2006). In addition to Ca²⁺ channel-dependent mechanisms that support vesicular fusion, intracellular Ca²⁺ signaling can also be initiated by receptor stimulation (Berridge, 1998). Endoplasmic reticulum-like structures and Ca²⁺-induced Ca²⁺ signaling are known to exist in presynaptic terminals (Bouchard et al., 2003; Verkhratsky, 2005) and IP3 receptor inhibition reduces NET activity. Thus, changes in presynaptic Ca²⁺/CaMK linked pathways may also be engaged as a consequence of presynaptic receptor stimulation and mobilization of intracellular Ca²⁺ stores. An example may be the movement of NETs triggered by angiotensin II in hindbrain noradrenergic neurons (Savchenko et al., 2003; Sumners and Raizada, 1986).

Finally, the NET mutants of the present invention and the trafficking pathway studied herein may be utilized to evaluate how psychotropic drugs modulate NET. Monoamine transporters are targets of psychostimulants including cocaine and amphetamines (Pacholczyk et al., 1991). Amphetamine alters the activities of NET and DAT via mechanisms requiring Ca²⁺, CaMK, and voltage-dependent Ca²⁺ channels (Kantor et al., 1999; Kantor et al., 2001; Kantor et al., 2004). Amphetamine triggers a rise in intracellular Ca²⁺ in NET expressing cells, and CaMK activation supports subsequent amphetamine-induced NET trafficking, suggesting that the psychostimulant may be manipulating pathways established for the regulated trafficking of the transporter. Dysregulation of these pathways may also support neuropsychiatric syndromes linked to altered NE signaling including anxiety, depression, post-traumatic stess disorder and attention-deficit disorder.

### II. SCREENING FOR NET ACTIVITY

Various methods are available for measuring the activity of a mutant NET of the present invention (e.g., in the presence or absence of a candidate NET modulator). In particular embodiments, the present invention provides a method for high throughput screening for modulators of the norepinepherine transporter. To accomplish this, a quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay in this invention is the use of cellular extracts that comprise a neurotransmitter. These may be cell membrane preparations that comprise a neurotransmitter transporter, particularly a norepinepherine transporter.

Another example is a cell-binding assay. While not directly addressing function, the ability of an inhibitor or blocker to bind to a target molecule (in this case the norepinepherine transporter) in a specific fashion is strong evidence of a related biological effect. For example, binding of a molecule to a norepinepherine transporter may, in and of itself, be inhibitory, due to steric, allosteric or charge-charge interactions. The norepinepherine transporter protein may be either free in solution, fixed to a support, expressed in or on the surface of a cell. Either the norepinepherine transporter or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

A. Measurement of Transport

In some embodiments, the present invention provides a novel and rapid method for analysis of transport by a norepinepherine transporter that comprises the measurement of uptake and/or accumulation of norepinepherine and analogues thereof that are specifically taken up by the transporter. Typically, this is accomplished by measuring the uptake or binding of radiolabeled norepinepherine (e.g. [³H]norepinepherine) or a radiolabeled antagonist. For example, uptake assays may be carried out in KRH/Ca²⁺ (mM: 120 NaCl, 4.7 KCl, 1.2 KH₂PO₄, 10 HEPES, 1.2 MgSO₄, 2.2 CaCl₂, pH 7.4,) with 0.1 mMs pargylnine, ascorbic acid, tropolone, and 1.8 mg/ml glucose. KRH/EGTA is KRH with 0.2 mM EGTA and without CaCl₂. Uptake assays may be initiated by addition of [³H]-NE (1-[7,8-³H] noradrenaline, Amersham Pharmacia). Nonspecific uptake may be evaluated using 1-10 μM desipramine. Assays may be carried out at 37° C. for 10 min in triplicates. Ca²⁺ imaging may be performed as described previously (Apparsundaram et al., 2001). CHO cells may be pre-loaded with 0.5 mM fura-2/acetoxymethyl ester (fura-2/AM, Molecular Probe) and superfused with KRH/EGTA and 1 μM thapsigargin for 10 min. The medium may be replaced with KRH/Ca²⁺ or KRH/EGTA before measurement of intracellular Ca²⁺.

1. Scintillation Proximity Assays

Measurement of transport may also be involve scintillation proximity assays, which is used to count the accumulated radiolabel on plates having scintillant embedded in them. Basically, cells may be plated at 50% confluence on 0.4-μm pore size 6.5-mm Transwell cell culture filter inserts and grown for 7 days. A cell monolayer growing on the porous membrane of the cell culture filter insert effectively separates each well in the cell culture plate into two chambers. The apical membranes of epithelial cells plated on these filters faces the chamber above the cells and the basolateral membranes face the lower chamber through the filter. After one wash each of the apical (upper chamber) and basolateral (lower chamber) sides of the monolayer with PBS/Ca/Mg, the cells are incubated in PBS/Ca/Mg containing ³H-labeled substrate either in the upper or the lower chamber at 22° C. At the end of the incubation, cells may be washed either three times from the apical side and once from the basolateral side (when ³H-labeled substrate was present in the upper chamber) or once from the apical side and three times from the basolateral side (when substrate was present in the lower chamber). The apical side of the cells may be washed by adding 0.2 ml of ice-cold PBS to the upper chamber and aspirating. The basolateral side of the cells may be washed by pipetting ice-cold PBS over the bottoms of the filter inserts. After the washes, the filters with cells attached may be excised from the insert cups, submerged in 3 ml of Optifluor scintillation fluid (Packard Instrument Co., Downers Grove, Ill.), and counted in a Beckman LS-3801 liquid scintillation counter. Transport assays on 48-well plates were described previously (Gu et al., 1994).

2. Voltage and Patch Clamp

The present invention also employs a means of determining the norepinepherine transporter activity or function by measuring the change in movement across a membrane, when the transporter is active. This may be accomplished using the voltage clamp technique, as is well known in the art, this allows the gating properties of the voltage-gated channels to be analyzed.

In short, the voltage clamp technique is a procedure whereby the transmembrane voltage of a membrane segment is rapidly set and maintained at a desired level. Once the membrane potential is controlled, the current flowing through the channels in that segment can be measured. The voltage clamp may be used to control the voltage of the membrane of an entire cell, i.e., as a “whole cell voltage clamp.”

The patch clamp technique allows the voltage clamp technique to be applied to a small patch of membrane containing very few voltage-sensitive channels. The basic idea behind a patch clamp experiment is to isolate a patch of membrane so small that it contains a single voltage-gated channel. Once this patch of membrane is isolated, the single channel can be voltage clamped. Using this technique, the gating properties of the norepinepherine transporter can be characterized.

B. Other Methods of Measurement of Transport

Other methods of measurement contemplated in the present invention may involve fluorescence microscopy. This may involve the use of fluorescent substrates, some of which are contemplated to be analogs of other native neurotransmitters.

1. Microscopy

Fluorescent microscopy is used to measure transport using norepinepherine or analogues thereof which are fluorescent substrates for the norepinepherine transporter. Cells that either endogenously or exogenously express a norepinepherine transporter are isolated and plated on glass bottom Petri-dishes or multi-well plates that may typically be coated with poly-L-lysine or any other cell adhesive agent. Cells are typically cultured for three or more days. The culture medium may then be aspirated and the cells are mounted on a Zeiss 410 confocal microscope. During the confocal measurement cells remain without buffer for approximately thirty seconds. Background autofluorescence can be established by collecting images for ten seconds prior to the addition of the buffer and norepinepherine or analogues thereof. As norepinepherine or an analogue thereof has a large Stoke shift between excitation (Iₘₐₓ=488 nm) and emission maxima (Iₘₐₓ=610 nm), the argon laser is tuned to 488 nm and the emitted light filtered with a 580-630 nm band pass filter (Iₘₐₓ=610 nm). The substantial red shift can be exploited to reduce background auto-fluorescence produced in the absence of substrate. The gain (contrast) and offset (brightness) for the photomultiplier tube (PMT) may be set to avoid detector saturation at the higher norepinepherine concentrations that may be used in certain experiments. The effects of photo-bleaching on norepinepherine accumulation may also be determined by examining the rate of norepinepherine accumulation and decay at various acquisition rates. In a constant pool of norepinepherine, rates as high as 20 Hz (50 msec/image) can be set.

In other embodiments, a fluorescent substrate may be used to measure transporter activity. Specifically, fluorescent substrate 4-(4-dimethylaminostyrl)-N-methylpyridinium (ASP+) is transported by NET and may be to measure neurotransmitter transport. U.S. Publication No. 20040115703. Further, 4-(4-(dimethylamino)phenyl)-1-methylpyridinium iodide (IDT307) is also transported by NET and may be used to measure neurotransmitter transport mechanisms using IDT307 and fluorescence microscopy (Blakely and DeFelice, 2007). Asp+ and/or IDT307 may be used to measure the activity of a NET mutant of the present invention in whole cell assays using plate fluorimetry (e.g., a FlexStation™ fluorimeter).

2. Fluorescence Anisotropy Measurements

To evaluate norepinepherine or analogues thereof binding to the surface membranes, cells expressing a norepinepherine transporter may be exposed to norepinepherine or analogues thereof with horizontal polarizer, with the polarizer rapidly switching to the vertical position. Cells may be imaged with alternating polarizations for 3 minutes to measure light intensity in the horizontal (Iₕ) and vertical (Iᵥ) positions in order to calculate the anisotropy ratio, r=(Iᵥ−gIₕ)/(Iᵥ+2 gIₕ). The factor g may be determined by using a half wave plate as described by Blackman et al. (1996). In this formulation, r=0.4 implies an immobile light source. Surface anisotropy can be measured at the cell circumference over 1 pixel width (0.625 mm). Cytosolic anisotropy can be measured near the center of the cell, approximately 5 pixel widths from the membrane.

3. Image Analysis

The fluorescent images may be processed using suitable software. For example, fluorescent images may be processed using MetaMorph imaging software (Universal Imaging Corporation, Downington Pa.). Fluorescent accumulation may be established by measuring the average pixel intensity of time resolved fluorescent images within a specified region identified by the DIC image. Average pixel intensity is used to normalize among cells.

4. Single Cell Fluorescence Microscopy

In some embodiments, the invention provides measurement of transporter characteristics at the single-cell level. Single-cell fluorescence microscopy provides a powerful assay to study rapid norepinepherine uptake kinetics from single cells.

5. Automation

The inventors further contemplate that all these methods are adaptable to high-throughput formats using robotic fluid dispensers, multi-well formats and fluorescent plate readers for the identification of norepinepherine transport modulators.

C. In Vivo Microdialysis

Microdialysis may be used in the present invention to monitor interstitial fluid in various body organs with respect to local metabolic changes. This technique may also be experimentally applied in humans for measurements in adipose tissue. In the present invention, the release of norepinepherine in the mouse brain, in response to stimuli may be analyzed using this technique.

Microdialysis procedure involves the insertion through the guide cannula of a thin, needle-like perfusable probe (CMA/12.3 mm×0.5 mm) to a depth of 3 mm in striatum beyond the end of the guide. The probe is connected beforehand with tubing to a microinjection pump (CMA−/100). The probe may be perfused at 2 μl/min with Ringer's buffer (NaCl 147 mM; KCl 3.0 mM; CaCl₂ 1.2 mM; MgCl₂ 1.0 mM) containing 5.5 mM glucose, 0.2 mM L-ascorbate, and 1 μM neostigmine bromide at pH 7.4). To achieve stable baseline readings, microdialysis may be allowed to proceed for 90 minutes prior to the collection of fractions. Fractions (20 μl) may be obtained at 10 minute intervals over a 3 hour period using a refrigerated collector (CMA170 or 200). Baseline fractions may be collected following the administration of a drug or a combination of drugs to be tested to the animal. Upon completion of the collection, each non-human animal (e.g., mouse) may be autopsied to determine accuracy of probe placement.

D. Evaluation of NET Phosphorylation

Phosphorylation of a NET may be evaluated by several methods known in art, such as using [³²P]. For phosphorylation, CHO-NET and CHO-NET T30A cells may be pre-incubated in phosphate free DMEM for 2 hours, and then incubated in phosphate free KBB (mMs: 25 NaHCO₃, 125 NaCl, 5 KCl, 5 MgSO₄, 10 glucose, pH7.3) with 1.5 mM CaCl₂ and carrier-free [³²P]-labeled orthophosphate (0.5 mCi/ml, Amersham) for 3 hours at 37° C. Cells may be briefly rinsed with KBB buffer with 0.2 mM EGTA and incubated in KBB/0.2 mM EGTA/carrier-free [³²P]-labeled orthophosphate (0.5 mCi/ml) for 15 min. At the end of incubation, CaCl₂ may be added into one set of cells at final concentration 2.2 mM, incubated for 5 min at RT. Cells may be washed with PBS/0.5 mM PMSF and lysed in PBS/1% TRITON X 100/0.5 mM PMSF/1 mM okadaic acid, 10 mM NaI, 1 mM Na orthovanadate, 10 mM Na pyruvate. Extracts may then be centrifuged at 16,000×g for 20 min, incubated with IgG coupled Sepharose (Amersham) for 30 min, unbound lysates were incubated with anti-HA agarose beads (Roche Applied Science) pre-blocked with non-labeled CHO cell lysates. Captured proteins by anti-HA beads can be separated using 3-12% linear gradient SDS/PAGE. Phosphorylated bands were captured via Phosphoimager (Typhoon 9400, Molecular Dynamics/GE Healthcare Life Sciences) and analyzed using ImageQuant 5.2 (Molecular Dynamics).

### III. NUCLEIC ACIDS ENCODING NET MUTANTS

Certain embodiments of the present invention concern a NET nucleic acid. In certain aspects, a NET nucleic acid comprises a wild-type or a mutant NET nucleic acid (e.g., comprising T30A or T30E, etc.). In particular aspects, a NET mutant nucleic acid encodes for or comprises a transcribed nucleic acid. In other aspects, a NET mutant nucleic acid comprises a nucleic acid segment of SEQ ID NO:1, SEQ ID NO:2, SEQ ID NO:4, or a biologically functional equivalent thereof. In particular aspects, a NET mutant nucleic acid encodes a protein, polypeptide, peptide.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from 1, 2, 3, 4, 5, to 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table non-limiting, purine and pyrimidine derivatives and analogs is also provided herein below.

TABLE 1
Purine and Pyrmidine Derivatives or Analogs

| Abbr. | Modified base description | Abbr. | Modified base description |
|---|---|---|---|
| ac4c | 4-acetylcytidine | Mam5s2u | 5-methoxyaminomethyl-2-thiouridine |
| Chm5u | 5-(carboxyhydroxylmethyl) uridine | Man q | Beta,D-mannosylqueosine |
| Cm | 2′-O-methylcytidine | Mcm5s2u | 5-methoxycarbonylmethyl-2-thiouridine |
| Cmnm5s2u | 5-carboxymethylamino-methyl-2- | Mcm5u | 5-methoxycarbonylmethyluridine |
| | thioridine | | |
| Cmnm5u | 5-carboxymethylaminomethyluridine | Mo5u | 5-methoxyuridine |
| D | Dihydrouridine | Ms2i6a | 2-methylthio-N6-isopentenyladenosine |
| Fm | 2′-O-methylpseudouridine | Ms2t6a | N-((9-beta-D-ribofuranosyl-2-methylthiopurine-6- |
| | | | yl)carbamoyl)threonine |
| Gal q | Beta,D-galactosylqueosine | Mt6a | N-((9-beta-D-ribofuranosylpurine-6-yl)N-methyl- |
| | | | carbamoyl)threonine |
| Gm | 2′-O-methylguanosine | Mv | Uridine-5-oxyacetic acid methylester |
| I | Inosine | o5u | Uridine-5-oxyacetic acid (v) |
| I6a | N6-isopentenyladenosine | Osyw | Wybutoxosine |
| m1a | 1-methyladenosine | P | Pseudouridine |
| m1f | 1-methylpseudouridine | Q | Queosine |
| m1g | 1-methylguanosine | s2c | 2-thiocytidine |
| m1I | 1-methylinosine | s2t | 5-methyl-2-thiouridine |
| m22g | 2,2-dimethylguanosine | s2u | 2-thiouridine |
| m2a | 2-methyladenosine | s4u | 4-thiouridine |
| m2g | 2-methylguanosine | T | 5-methyluridine |
| m3c | 3-methylcytidine | t6a | N-((9-beta-D-ribofuranosylpurine-6- |
| | | | yl)carbamoyl)threonine |
| m5c | 5-methylcytidine | Tm | 2′-O-methyl-5-methyluridine |
| m6a | N6-methyladenosine | Um | 2′-O-methyluridine |
| m7g | 7-methylguanosine | Yw | Wybutosine |
| Mam5u | 5-methylaminomethyluridine | X | 3-(3-amino-3-carboxypropyl)uridine, (acp3)u |

A nucleobase may be comprised in a nucleside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

C. Nucleotides

As used herein, a “nucleotide” refers to a nucleoside further comprising a “backbone moiety.” A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as flourescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance, cellular uptake and regulating RNA expression; U.S. Pat. No. 5,858,988 which describes hydrophobic carrier agent attached to the 2′-O position of oligonucleotides to enhanced their membrane permeability and stability; U.S. Pat. No. 5,214,136 which describes olignucleotides conjugaged to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

E. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5,891,625. Other modifications and uses of nucleic acid analogs are known in the art, and are encompassed by the NET mutant. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. U.S. application Ser. No. 117,363 describes several alkylamino moeities used to enhance cellular uptake of a PNA. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

F. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

G. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

H. Nucleic Acid Segments

In certain embodiments, the nucleic acid is a nucleic acid segment. As used herein, the term “nucleic acid segment,” are smaller fragments of a nucleic acid, such as for non-limiting example, those that encode only part of the NET mutant peptide or polypeptide sequence. Thus, a “nucleic acid segment” may comprise any part of a gene sequence, of from about 2 nucleotides to the full length of the NET mutant peptide or polypeptide encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

I. Nucleic Acid Complements

The present invention also encompasses a nucleic acid that is complementary to a NET mutant nucleic acid. In particular embodiments the invention encompasses a nucleic acid or a nucleic acid segment complementary to the sequence set forth in SEQ ID NO:1, SEQ ID NO:2, or SEQ ID NO:4. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

J. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02M to about 0.15M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15M to about 0.9M NaCl at a temperature range of about 20° C. to about 50° C. Of course, it is within the skill of one in the art to further modify the low or high stringency conditions to suite a particular application.

### IV. NUCLEIC ACID-BASED EXPRESSION SYSTEMS

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 2 lists non-limiting examples of elements/promoters that may be employed, in the context of the present invention, to regulate the expression of a RNA. Table 3 provides non-limiting examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin Heavy Chain | Banerji et al., 1983; Gilles et al., 1983; Grosschedl et |
| | al., 1985; Atchinson et al., 1986, 1987; Imler et al., |
| | 1987; Weinberger et al., 1984; Kiledjian et al., 1988; |
| | Porton et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn |
| | et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase | Jaynes et al., 1988; Horlick et al., 1989; Johnson et al., |
| (MCK) | 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsch et al., 1990 |
| Molecule (NCAM) | |
| α₁-Antitrypsin | Latimer et al., 1990 |
| H2B (TH2B) Histone | Hwang et al., 1990 |
| Mouse and/or Type I | Ripe et al., 1989 |
| Collagen | |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth | Pech et al., 1989 |
| Factor (PDGF) | |
| Duchenne Muscular | Klamut et al., 1990 |
| Dystrophy | |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al., 1980; |
| | Katinka et al., 1980, 1981; Tyndell et al., 1981; Dandolo |
| | et al., 1983; de Villiers et al., 1984; Hen et al., 1986; |
| | Satake et al., 1988; Campbell and/or Villarreal, 1988 |
| Retroviruses | Kriegler et al., 1982, 1983; Levinson et al., 1982; |
| | Kriegler et al., 1983, 1984a, b, 1988; Bosze et al., 1986; |
| | Miksicek et al., 1986; Celander et al., 1987; Thiesen |
| | et al., 1988; Celander et al., 1988; Choi et al., 1988; |
| | Reisman et al., 1989 |
| Papilloma Virus | Campo et al., 1983; Lusky et al., 1983; Spandidos |
| | and/or Wilkie, 1983; Spalholz et al., 1985; Lusky et al., |
| | 1986; Cripe et al., 1987; Gloss et al., 1987; Hirochika |
| | et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency | Muesing et al., 1987; Hauber et al., 1988; Jakobovits |
| Virus | et al., 1988; Feng et al., 1988; Takebe et al., 1988; |
| | Rosen et al., 1988; Berkhout et al., 1989; Laspia et al., |
| | 1989; Sharp et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1987b; McNeall |
| | | et al., 1989 |
| MMTV (mouse | Glucocorticoids | Huang et al., 1981; Lee et al., |
| mammary tumor virus) | | 1981; Majors et al., 1983; |
| | | Chandler et al., 1983; Lee et al., |
| | | 1984; Ponta et al., 1985; Sakai et al., 1988 |
| β-Interferon | Poly(rI) × | Tavernier et al., 1983 |
| | Poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H- | Interferon | Blanar et al., 1989 |
| 2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis Factor α | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

8. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

9. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

10. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention include adenoviral vectors, AAV vectors, retroviral vectors, vaccinia virus (Ridgeway, 1988; Baichwal and Sugden, 1986; Coupar et al., 1988), sindbis virus, cytomegalovirus and herpes simplex virus.

B. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

1. Ex Vivo Transformation

Methods for transfecting vascular cells and tissues removed from an organism in an ex vivo setting are known to those of skill in the art. For example, cannine endothelial cells have been genetically altered by retrovial gene transfer in vitro and transplanted into a canine (Wilson et al., 1989). In another example, yucatan minipig endothelial cells were transfected by retrovirus in vitro and transplanted into an artery using a double-ballonw catheter (Nabel et al., 1989). Thus, it is contemplated that cells or tissues may be removed and transfected ex vivo using the nucleic acids of the present invention. In particular aspects, the transplanted cells or tissues may be placed into an organism. In preferred facets, a nucleic acid is expressed in the transplanted cells or tissues.

2. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of NET mutant used may vary upon the nature of the antigen as well as the organelle, cell, tissue or organism used

3. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 9217598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

4. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

5. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

6. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

7. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

8. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

C. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

A tissue may comprise a host cell or cells to be transformed with a NET mutant. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, anthers, ascite tissue, cobs, ears, flowers, husks, kernels, leaves, meristematic cells, pollen, root tips, roots, silk, stalks, and all cancers thereof.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art (see, for example, phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

D. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE's COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g., 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

### V. NET POLYPEPTIDES

Various aspects of the present invention relate to a purified or substantially purified NET mutant polypeptide, e.g., a NET comprising a substitution mutation at T30. The term “purified proteins, polypeptides, or peptides” as used herein, is intended to refer to an proteinaceous composition, isolatable from mammalian cells or recombinant host cells, wherein the at least one protein, polypeptide, or peptide is purified to any degree relative to its naturally-obtainable state, i.e., relative to its purity within a cellular extract. A purified protein, polypeptide, or peptide therefore also refers to a wild-type or mutant protein, polypeptide, or peptide free from the environment in which it naturally occurs.

Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as described herein below, or as would be known to one of ordinary skill in the art for the desired protein, polypeptide or peptide.

Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. In addition to those techniques described in detail herein below, various other techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of a specific fusion protein using a specific binding partner. Such purification methods are routine in the art. As the present invention provides DNA sequences for the specific proteins, any fusion protein purification method can now be practiced. This is exemplified by the generation of an specific protein-glutathione S-transferase fusion protein, expression in E. Coli, and isolation to homogeneity using affinity chromatography on glutathione-agarose or the generation of a polyhistidine tag on the N- or C-terminus of the protein, and subsequent purification using Ni-affinity chromatography. However, given many DNA and proteins are known, or may be identified and amplified using the methods described herein, any purification method can now be employed.

Although preferred for use in certain embodiments, there is no general requirement that the protein, polypeptide, or peptide always be provided in their most purified state. Indeed, it is contemplated that less substantially purified protein, polypeptide or peptide, which are nonetheless enriched in the desired protein compositions, relative to the natural state, will have utility in certain embodiments.

Methods exhibiting a lower degree of relative purification may have advantages in total recovery of protein product, or in maintaining the activity of an expressed protein. Inactive products also have utility in certain embodiments, such as, e.g., in determining antigenicity via antibody generation.

A. Biological Equivalents

In certain embodiments, a biological equivalent of a NET mutant may be used with the present invention. The biological functional equivalent may comprise a polynucleotide that has been engineered to contain distinct sequences while at the same time retaining the capacity to encode the “wild-type” or standard protein. This can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a protein.

In another example, a polynucleotide made be (and encode) a biological functional equivalent with more significant changes. Certain amino acids may be substituted for other amino acids in a protein structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) in selected amino acids (or codons) may be substituted. Functional activity is defined as the phosphorylation state and/or the trafficking of a NET due to the particular amino acid at position 30 (e.g., T30, etc.).

In general, the shorter the length of the molecule, the fewer changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and/or arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

The present invention, in many aspects, relies on the synthesis of peptides and polypeptides in cyto, via transcription and translation of appropriate polynucleotides. These peptides and polypeptides will include the twenty “natural” amino acids, and post-translational modifications thereof. However, in vitro peptide synthesis permits the use of modified and/or unusual amino acids.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Antibodies, materials, siRNA and cDNA constructs. Anti-hemagglutinin (HA) antibody (3F10) conjugated with peroxidase (Roche), anti-CaMKI (Santa Cruz Biotechnology), anti-CaMKIIδ (Santa Cruz Biotechnology), and anti-transferrin receptor (Zymed) were used at a dilution of 1:500, 1:100, 1:20, and at 3 μg/ml, respectively for immunoblots. NET 17-1 antibody (Mab Technologies) was used at 1 μg/ml for immunoblotting. Anti-NET sera 43408 detecting an epitope at the extracellular loop of NET have been described previously (Savchenko et al., 2003) and used at 1:500 for immunohistochemistry. Bisindolylmalemide I (BIM) was from Calbiochem, BAPTA/AM, thapsigargin, KN-93, and W7 from Alexis, and STO-609 from Tocris. Desipramine, verapamil, EGTA (ethylene glycol-bis(b-aminoethyl ether) N,N,N′,N′-tetraacetic acid), and all other chemicals were from Sigma. siRNAs for CaMKI and CaMKIIδ (SMARTpool™) were purchased from Dharmacon. cDNA constructs for human NET and its mutants (NET Δ28-47, NET T30A, NET T30E) were N-terminally tagged by inserting HA-tag (YPYDVPDYA) between the first and second amino acids.

Cell culture, transfection of cDNA or siRNA, generation of stable cell lines, preparation of cortical synaptosomes. CHO cells were maintained in DMEM/10% fetal bovine serum (FBS), 2 mM L-glutamine (L-Glu), 100 IU/ml penicillin, 100 μg/ml streptomycin (pen/strep). CHO-NET or CHO-NET T30A cells were generated by stably transfecting HA-NET or HA-NET T30A in pcDNA5/FRT (Invitrogen) into CHO-Flip-In cells (Invitrogen). The stable clones were selected using hygromycin B (500 mg/ml, Invitrogen) and maintained in Ham's F12/10% FBS/L-Glu/pen/strep supplemented with Zeocin 100 mg/ml (Invitrogen). CAD-NET cells were generated by stably transfecting HA-NET in pcDNA3 into CAD cells and maintained in DMEM/F12/8% FBS/L-Glu/pen/strep/200 μg/ml of G418 (Mediatech). TransIT-LT1 transfection reagent (Mirus) was used for all transfections. All cells were plated on poly-D-lysine coated plates and incubated for 48 hrs for cDNA transfection and for 48 to 72 hrs for siRNA transfection prior to assays. For synaptosomal preparations, brain cortex was dissected from mice (C57/B16, Harlan) and homogenized in 10 mM HEPES, 0.32 M Sucrose, pH 7.4 using a Teflon pestle/homogenizer. Homogenates were centrifuged at 1,000 g, 5 min at 4° C., and then the supernatant were re-centrifuged at 16,000 g, 20 min at 4° C. The pellets were collected as synaptosomes.

Assay buffers, transport assays, Ca²⁺ imaging. NE transport assays on synaptosomes (50-100 μg/assay reaction) or cells were described previously (Sung et al., 2003). Uptake assay was carried out in KRH/Ca²⁺ (mM: 120 NaCl, 4.7 KCl, 1.2 KH₂PO₄, 10 HEPES, 1.2 MgSO₄, 2.2 CaCl₂, pH 7.4,) with 0.1 mM pargylnine, ascorbic acid, tropolone, and 1.8 mg/ml glucose, otherwise mentioned. KRH/EGTA is KRH with 0.2 mM EGTA and without CaCl₂. When KRH/EGTA was used, KRH/Ca²⁺ for the parallel experiment contained 0.2 mM EGTA as well. Uptake assays were initiated by addition of [³H]-NE (1-[7,8-³H] noradrenaline, Amersham Pharmacia) at 50 nM final concentration. For kinetics assays, 50, 100, 200 nM of [³H]-NE, 400, 600, 800, 1,000, 1,200, 1,500 nM of 20% [³H]-NE and 80% of unlabeled NE (Sigma) were used. Nonspecific uptake was defined using 1-10 μM desipramine. All assays were carried out at 37° C. for 10 min in triplicates. Ca²⁺ imaging was performed as described previously (Apparsundaram et al., 2001). CHO cells were pre-loaded with 0.5 mM fura-2/acetoxymethyl ester (fura-2/AM, Molecular Probe) and superfused with KRH/EGTA and 1 μM thapsigargin for 10 min. The medium was replaced with KRH/Ca²⁺ or KRH/EGTA before measurement of intracellular Ca²⁺. All experiments derive from at least 3 independent data and mean values were evaluated using a two-tailed Student's t-test or a one-way ANOVA, followed by Tukey's test, with p<0.05 considered significant (*). Data were analyzed using GraphPad Prism 4.

Protocols for restoration and depletion of Ca²⁺ For uptake assay, synaptosomes were pre-incubated with drugs in KRH/Ca²⁺. For increase of Ca²⁺, synaptosomes were re-suspended in KRH/EGTA, transferred to fresh KRH/EGTA (EGTA to EGTA) or KRH/Ca²⁺ (EGTA to Ca²⁺), and incubated at 37° C. for 5 min prior to the addition of radiolabeled NE. NE uptake activity of synaptosomes in KRH/Ca²⁺ was compared to the activity of synaptosomes in KRH/EGTA. For reduction of Ca²⁺, synaptosomes were first resuspended in KRH/Ca²⁺, divided into 2 aliquots, replaced with fresh KRH/Ca²⁺ (Ca²⁺ to Ca²⁺) or KRH/EGTA (Ca²⁺ to EGTA), and incubated at 37° C. for 5 min prior to the addition of radiolabeled NE. NE uptake activity of synaptosomes in KRH/EGTA was compared to the activity of synaptosomes in KRH/Ca²⁺. Control cells were treated the same way except use of complete media with vehicle. Restoration of Ca²⁺ for surface biotinylation was carried out by incubating cells in KRH/EGTA with 0.1-1 μM thapsigargin for 10 min at 37° C. to deplete Ca²⁺. Cells were washed once with KRH/EGTA, replaced with KRH/EGTA or KRH/Ca²⁺, and incubated for 1 min at RT to 5 min at 37° C. prior to biotinylation. Ca²⁺ depletion was carried out by incubating cells in complete medium with 10 mM EGTA/30 mM BAPTA/AM (CHO cells) or complete medium with 10 mM EGTA (CAD cells) for 1 min at RT or for 5-10 min at 37° C. prior to biotinylation.

Primary neuronal culture, immunohistochemistry, electrophysiology. Superior cervical ganglia (SCG) were dissected from postnatal day 1-3 pups of Sprague-Dawley rats (Harlan) and the sympathetic neurons were cultured as described previously (Savchenko et al., 2003) in UltraCulture medium (BioWhittaker) supplemented with nerve growth factor (20 ng/ml; Sigma), 3% FBS, 2 mM L-glutamine, penicillin (100 units/ml), streptomycin (100 μg/ml) at 37° C. in humidified 5% CO₂ for 9-14 days. Surface-labeling of NET has been preformed by staining non-fixed SCG culture with anti-NET sera 43408 as described in (Savchenko et al., 2003). For detection of plasma membrane changes+/−depolarization, SCG neurons were preincubated with normal medium (2.5 mM K+) or in 40 mM or 90 mM K⁺ for 15 min followed by incubation with NET 43408 antibody for 1 hr at RT in the absence of detergent. Cells were washed and fixed with 3% p-formaldehyde for 10 min prior to visualization of NET labeling using Cy3-conjugated anti-rabbit antibodies. Specimens from multiple fields were examined with a Zeiss LSM 510 Meta imaging system equipped with internal He/Ne and external Ar/Kr lasers. Images were collected were averaged across multiple fields of replicate experiments (n=3), pseudocolored for presentation, and pixel intensity calculated over processes immunopositive by double-staining for tubulin immunoreactivity in Metamorph.

Whole Cell Patch Clamp Recording of NET currents. Patch clamp experiments were performed using an amplifier Axopatch 200B with a low-pass filter set at 1 kHz. Quartz patch pipettes with 5-7 MΩ resistance were pulled by a programmable puller (model P-2000, Sutter Instruments Novato, Calif.) and filled with the internal solution containing (in mM): 120 KCl, 2 MgCl₂, 10 HEPES, 2 MgATP, 30 dextrose and adjusted to pH 7.35. SCG neurons were washed twice with a control solution containing mMs 130 NaCl, 5 CaCl₂, 0.5 MgCl₂, 1.3 KCl, 10 HEPES, 34 dextrose adjusted to pH 7.35 before experiments. NET-mediated current was defined as the current recorded in control condition minus the current recorded in presence of 5 μM desipramine. Neurons were clamp at −50 mV and the NET-mediated current was recorded by stepping the membrane voltage to −120 mV for 500 ms before and after a 2 sec depolarizing step at −10 mV. NET-mediated current was studied in the control condition, in presence of 200 μM CdCl₂, after 15 min preincubation with 5 μM KN93 and after 20 min preincubation with 2 μM STO609.

Biochemical analysis and phosphorylation. Cell surface biotinylation was performed as described previously (Sung et al., 2003). For phosphorylation, CHO-NET and CHO-NET T30A cells were pre-incubated in phosphate free DMEM for 2 hours, and then incubated in phosphate free KBB (mMs: 25 NaHCO₃, 125 NaCl, 5 KCl, 5 MgSO₄, 10 glucose, pH7.3) with 1.5 mM CaCl₂ and carrier-free [³²P]-labeled orthophosphate (0.5 mCi/ml, Amersham) for 3 hours at 37° C. Cells were briefly rinsed with KBB buffer with 0.2 mM EGTA and incubated in KBB/0.2 mM EGTA/carrier-free [³²P]-labeled orthophosphate (0.5 mCi/ml) for 15 min. At the end of incubation, CaCl₂ was added into one set of cells at final concentration 2.2 mM, incubated for 5 min at RT. Cells were washed with PBS/0.5 mM PMSF and lysed in PBS/1% TRITON X 100/0.5 mM PMSF/1 mM okadaic acid, 10 mM NaI, 1 mM Na orthovanadate, 10 mM Na pyruvate. Extracts were centrifuged at 16,000×g for 20 min, incubated with IgG coupled Sepharose (Amersham) for 30 min, unbound lysates were incubated with anti-HA agarose beads (Roche Applied Science) pre-blocked with non-labeled CHO cell lysates. Captured proteins by anti-HA beads were separated using 3-12% linear gradient SDS/PAGE. Phosphorylated bands were captured via Phosphoimager (Typhoon 9400, Molecular Dynamics/GE Healthcare Life Sciences) and analyzed using ImageQuant 5.2 (Molecular Dynamics). All other biochemical methods are described previously (Sung et al., 2003). Protein electrophoresis was performed using 10% SDS/PAGE except for phosphorylation studies. Exposed films of immunoblots were scanned using an Agfa Duoscan T1200 and the captured images processed in Adobe® Photoshop® and quantitated using NIH image.

### Example 2

### External Ca²⁺ Alterations Modulate NE Transport

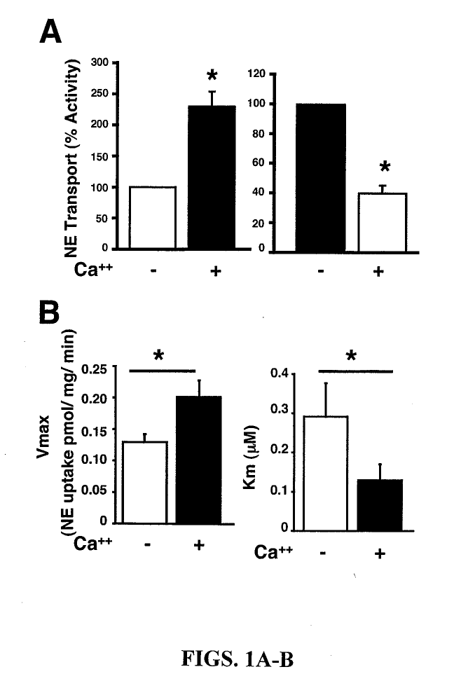

In order to gauge the sensitivity of presynaptic NET activity to changes in Ca²⁺ external Ca²⁺ was manipulated in mouse cortical synaptosomal preparations. As shown in FIG. 1A (Left panel), synaptosomes incubated in the absence of Ca²⁺ (KRH/EGTA) (see Methods) demonstrate a significant elevation (267+/−19.4% versus vehicle) in NE transport when supplemented with Ca²⁺ (2.2 mM Ca²⁺ final) 5 min prior to transport assays. This regulation appeared reversible as synaptosomes prepared by the same way, but first incubated in KRH/Ca²⁺ and then switched to KRH/EGTA demonstrated a reduction to 38.33+/−4.25% in NE transport relative to activity measured in synaptosomes maintained in KRH/Ca²⁺ (FIG. 1A, right). NE transport saturation analyses conducted in KRH/EGTA or KRH/Ca²⁺ revealed that these activity changes arise from a significant increase in Vmax (0.13+/−0.01 pmol/mg protein/min in KRH/EGTA versus 0.20+/−0.03 pmol/mg protein/min in KRH/Ca²⁺, FIG. 1B, left) as well as a reduced NE Km (0.29+/−0.08 nM in KRH/EGTA versus 0.13+/−0.04 nM in KRH/Ca²⁺, FIG. 1B, right).

### Example 3

### CaMKs Support Ca²⁺-Dependent NE Transport

The contribution of Ca²⁺ linked kinases to Ca²⁺ sensitive NET activity in synaptosomes was evaluated next. Treatments of synaptosomes with bisindolylmaleimide I (BIM; a PKC inhibitor), KN93 (a CaMK inhibitor), and W7 (a calmodulin antagonist) each inhibited NE transport in KRH/Ca²⁺ (FIG. 2A). The inventors also examined whether pre-incubation with these antagonists influence the changes in NET activity arising from alterations in medium Ca²⁺. As shown in FIG. 2B, KN93 significantly blunted the elevation in NET activity triggered by addition of Ca²⁺ (2.2 mM Ca²⁺ final) to KRH/EGTA medium. Addition of Ca²⁺ increased NE transport of vehicle-treated synaptosomes to 242.8+/−23.4% of control, but increased only to 140.5+/−11.2% of control in KN93 pre-treated synaptosomes. In contrast, BIM demonstrated no ability to attenuate Ca²⁺-stimulated NET activity even at doses that diminish basal NE transport (FIG. 2C). Additionally, the reduction in NET activity that occurs when synaptosomes are switched from KRH/Ca²⁺ to KRH/EGTA is blunted by KN93. Thus, synaptosomes treated with KN93 exhibited 67.5+/−7.2% of the NE uptake observed in KRH/Ca²⁺ when switched to KRH/EGTA as compared to a drop to 38.3+/−4.2% of control (FIG. 2D) with vehicle treatment. As with Ca²⁺ supplementation, BIM incubations failed to attenuate the loss of NET activity in the absence of Ca²⁺ (FIG. 2E). These findings point to a dominant role of CaMKs over BIM-sensitive PKCs in Ca²⁺-dependent NET activity.

### Example 4

### Ca²⁺-Dependent Surface Trafficking of NET in Transfected Cells

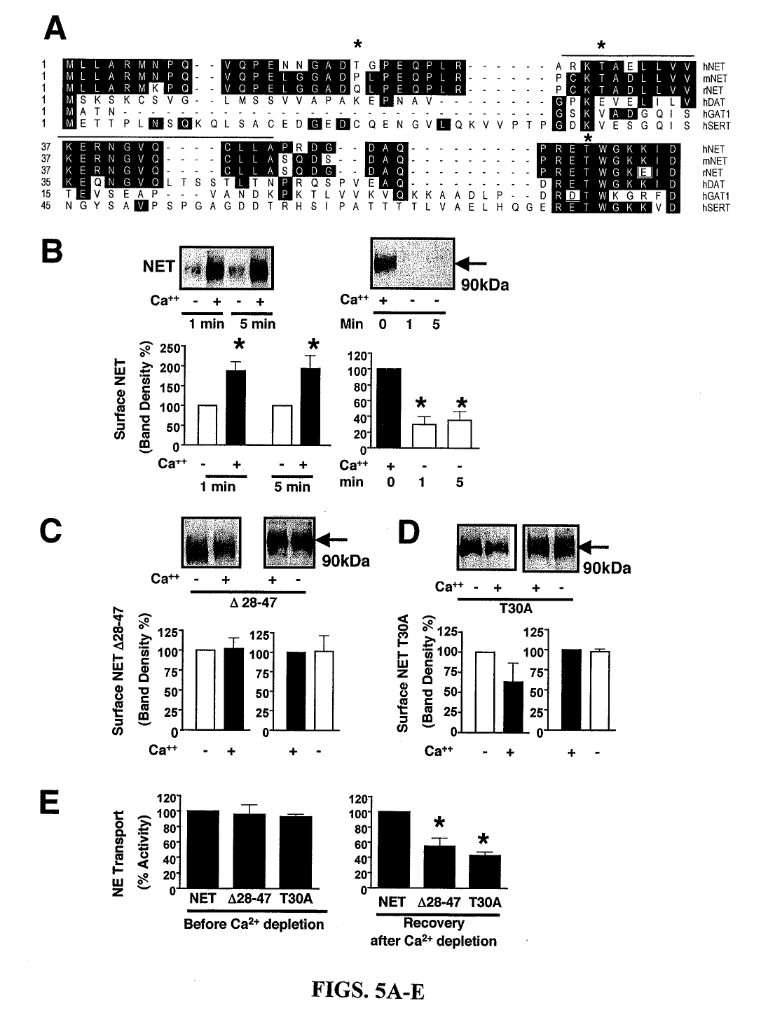

NET is expressed at low density in brain synaptosomes, precluding extensive biochemical studies using available reagents. To advance studies of NET regulation in a biochemically more tractable system, the inventors tested whether Ca²⁺ regulation of NE transport is linked to Ca²⁺ induced surface trafficking of NET in transfected cells. As previously documented, transfected Chinese Hamster Ovary (CHO) cells are a suitable vehicle for monitoring syntaxin 1A modulation of NET (Sung et al., 2003). Although CHO cells lack expression of voltage-sensitive Ca²⁺ channels, cytoplasmic Ca²⁺ in these cells can be rapidly elevated through manipulation of external Ca²⁺ concentrations after store depletion (Fagan et al., 1996; Gailly, 1998). When Ca²⁺-depleted, NET-transfected CHO cells were replaced with KRH/Ca²⁺, CHO cells exhibit a rapid Ca²⁺ influx that peaks 1 min after alteration of external Ca²⁺ influx with a gradual decline to baseline in 10 min, as detected by Fura-2 based Ca²⁺ imaging (FIG. 3A). Ca²⁺ influx under these conditions supports a significant elevation in NE transport comparable to changes observed in brain synaptosomes and in NET surface number (FIG. 3B, left). In contrast, Ca²⁺-depletion significantly reduced NET surface number (FIG. 3B, right). These changes in transport and surface density are rapid, with significant changes detectable within 1 min following Ca²⁺ manipulations (also see FIG. 5), and do not arise from changes in total NET protein. In contrast, neither Ca²⁺ elevations nor Ca²⁺-depletion resulted in consistent trafficking responses of transferrin receptors. Importantly, and consistent with studies in synaptosomes, the inventors found that KN93 blocked both the Ca²⁺-stimulated elevations as well as the Ca²⁺-depletion elicited reductions in NET surface density (FIG. 3B, left and right).

KN93 is known to inhibit CaMKI, CaMKII, and CaMKIV (Hook and Means, 2001). Because CaMKIV expression and function is restricted to the nucleus (Hook and Means, 2001), further analyses focused on CaMKI and CaMKII. Whereas CaMKII can be directly activated by Ca²⁺ and calmodulin, CaMKI requires activation/phosphorylation by CaMK kinase (CaMKK) (Hook and Means, 2001). To test a role for CaMKI, transfected CHO cells were preincubated with the CaMKK inhibitor STO-609 (Tokumitsu et al., 2002; Wayman et al., 2004), prior to altering Ca²⁺ content of the culture medium (FIG. 3C). Pre-incubation of CHO cells with STO-609 completely blocked the increase in surface NET protein evident in vehicle treated cells shifted from KRH/EGTA to KRH/Ca²⁺ (FIG. 3C left). Conversely, preincubation with STO-609 also prevented a loss of NET from the surface upon Ca²⁺ depletion (FIG. 3C right). These findings suggest that CaMKI and/or CaMKII support Ca²⁺-dependent NET surface expression in transfected CHO cells.

### Example 5

### Suppression of Neuronal CaMKI and CaMKII Attenuates Ca²⁺ Regulation of NET

To extend findings with pharmacological inhibition of CaMKs, a mouse neuronal cell model was used where CaMKs could be manipulated using RNA interference. Specifically, noradrenergic CAD cells (Sung et al., 2003), stably expressing HA-tagged NET (CAD-NET) were used. CAD cells express CaMKI and CaMKIIδ (Donai et al., 2000). In transfected CAD cells, Ca²⁺ regulation of NE transport similar to CHO cells and cortical synaptosomes was observed as well as a loss of NE transport activity when inhibiting L-type voltage dependent Ca²⁺ channels, the predominant isoform in this cell line, with verapamil (Wang and Oxford, 2000). Immunoblots of CAD-NET cell extracts indicate that CaMK siRNAs significantly down-regulated targeted kinase expression without significant effects on NET protein expression (FIG. 4A). Furthermore, in cells with suppressed CaMKII expression, a reduction in basal NE transport was detected (transport in KRH/Ca²⁺ as well as a loss of sensitivity to KN93 (FIG. 4B). In contrast, CamKI siRNAs only exerted a small reduction in basal NE uptake that retained KN93 sensitivity (FIG. 4B).

Next the inventors asked whether Ca²⁺-dependent changes in NET surface expression are sensitive to siRNA-mediated suppression of CaMKs (FIG. 4C). For these experiments, the inventors focused on Ca²⁺ depletion as loss of regulation in this paradigm generates a positive signal for surface expression. As in CHO cells, the 90 kDa form of NET predominates at the surface (FIG. 4C). Whereas Ca²⁺ depletion of mock-transfected CAD-NET cells reveals the expected reduction in surface NET, cells transfected with CaMKI siRNA retained NET at the cell surface after Ca²⁺ depletion (FIGS. 4C and 4D). Consistent with observations in NE transport assays (FIG. 4B), CaMKII siRNA transfections also appear to reduce basal surface NET expression though this effect is somewhat less consistent (FIGS. 4C & D). Importantly, CamKII siRNA blunted the trafficking response to removal of Ca²⁺ (FIGS. 4C and 4D).

### Example 6

### The NET NH₂ Terminus is Responsible for Ca²⁺ Triggered Surface Trafficking

Using transfected NET, the inventors initiated an analysis of structural determinants in the transporter that are required for Ca²⁺-dependent surface trafficking. Here the inventors focused on the NET NH₂ terminus as this domain contains an interaction site with syntaxin 1A, a protein that participates in NET surface trafficking (Sung et al., 2003) and which exhibits Ca²⁺-dependent NET associations (Sung and Blakely, submitted). The NET NH₂ terminus is relatively divergent from other biogenic amine transporters (FIG. 5A), particularly as compared to transmembrane domains, and the inventors hypothesized that this domain might possess unique structural or sequence motifs that support Ca²⁺ regulation. In the course of prior analyses of NET NH₂ terminal deletions affecting NET transport and protein associations (Sung et al., 2003), the inventors created a deletion spanning amino acids 28 to 47 (NETΔ28-47). Whereas NETΔ28-47 expresses mature protein at levels comparable to wild-type NET, the inventors found that the mutant displays a striking loss of sensitivity to Ca²⁺ manipulations. Thus, whereas wild-type NET supports changes in surface expression within a min following Ca²⁺ addition or depletion (FIG. 5B), surface expression of NETΔ28-47 did not respond to either Ca²⁺ addition (FIG. 5C, left) or Ca²⁺ depletion (FIG. 5C, right).

As Ca²⁺ elicited surface trafficking of NET requires CaMKs, the inventors examined the region encompassed by NET amino acids 28-47 with respect to potential sites of Ser/Thr phosphorylation. Human NET contains no Ser residues in the NH₂ terminus but does possess 3 Thr residues, T19, T30, and T58 that could serve as phosphorylation sites (FIG. 5A). Of these residues, T19 is not conserved among NETs, T30 is conserved among, and is unique to, NETs among biogenic amine transporters. Although T58 is conserved across all monoamine transporters, this residue, like T19, lies outside the 28-47 deletion and these residues were therefore not considered high-priority candidates. The inventors therefore mutated T30 to investigate its contribution to Ca²⁺ regulation of NET surface trafficking. NET T30A, like NETΔ28-47, exhibits normal transporter protein expression (NETΔ28-47 and T30A basal surface expression=106+/−18% and 117+/−18.5% respectively versus wildtype hNET, n=3). However, NET T30A displays a complete lack of Ca²⁺ sensitivity with respect to expected Ca²⁺-dependent changes in transporter surface density (FIG. 5D) regardless of the direction of Ca²⁺ manipulations.

Consistent with surface expression findings, NETΔ28-47 and NET T30A display NE transport activity equivalent to wild-type NET when measured in KRH/Ca²⁺ (FIG. 5E Left). Although protein levels did not respond to changes in Ca²⁺, the inventors did note a loss of uptake with activity with Ca²⁺ depletion and a difference with respect to recovery of NE transport after 15 min period of Ca²⁺ restoration including the 10 min transport assay (FIG. 5E Right), where both NETΔ28-47 and NET T30A failed to recover NE transport activity as observed for wild-type NET. These results indicate that the normal translation of intracellular Ca²⁺ variation to changes in NE uptake are thwarted by the T30A mutation.

### Example 7

### T30 Supports Ca²⁺-Induced Phosphorylation of NET

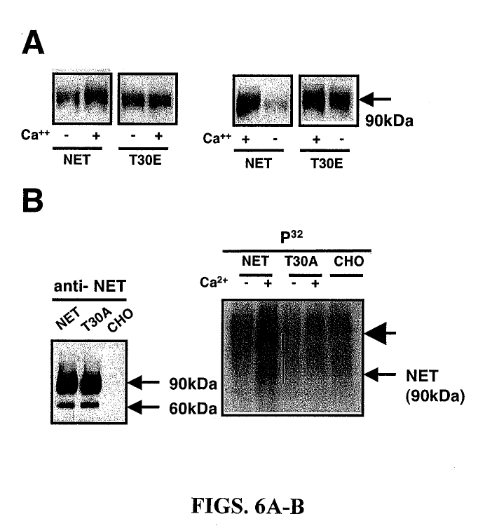

To further investigate a role for T30 in Ca²⁺-dependent NET surface expression, the inventors engineered NET T30E, a mutant designed to mimic constitutive phosphorylation at T30. NET T30E expresses and matures like NET, but, like NET T30A, NET T30E surface density failed to change in response to Ca²⁺ manipulations whether an increase or depletion of medium Ca²⁺ was effected (FIG. 6A).

Next, the inventors asked whether changes in Ca²⁺ that elevate NET surface expression in CHO cells can trigger phosphorylation of NET and whether T30 is important for phosphorylation. For this experiment, the inventors performed immunoprecipitations of metabolically [³²P]-labeled CHO cells stably expressing NET or NET T30A at the same genomic locus (see Materials and Methods). These cell lines express equivalent amounts of NET proteins (FIG. 6B, left) and also transport NE equivalently. Switching cells in KRH/EGTA to KRH/Ca²⁺ for 5 min prior to immunoprecipitation for NET induces phosphorylation of proteins that migrate on SDS-PAGE at ˜90 kDa which parallels the migration of mature, surface NET surface protein (lower arrow, FIG. 6B, right). Additionally, the inventors reproducibly recovered a phosphorylated species that migrates at 150-200 kDa (upper arrow, FIG. 6B, right). As the latter band does not comigrate with a species exhibiting NET immunoreactivity (FIG. 6B, left), these findings suggest that addition of Ca²⁺ to Ca²⁺-free medium not only induced phosphorylation of NET but also that NET likely exists as a complex with unidentified Ca²⁺-sensitive phosphoproteins. Importantly, similar immunoprecipitations with NET T30A-transfected cells revealed no phosphorylation of either the transporter or the higher mass species following Ca²⁺ additions. Finally, the inventors tested the impact of other serine/threonine residues resident in NET consensus CaMKII-phosphorylation sites including T58, T238, S259, S502, S579, T580, S583. All of these mutants retained sensitivity to Ca²⁺ manipulations. This data indicates that T30 has a unique and essential role in translation of intracellular Ca²⁺ changes to changes in NET surface expression linked to transporter phosphorylation.

### Example 8

### Ca²⁺ Supports NET Surface Trafficking

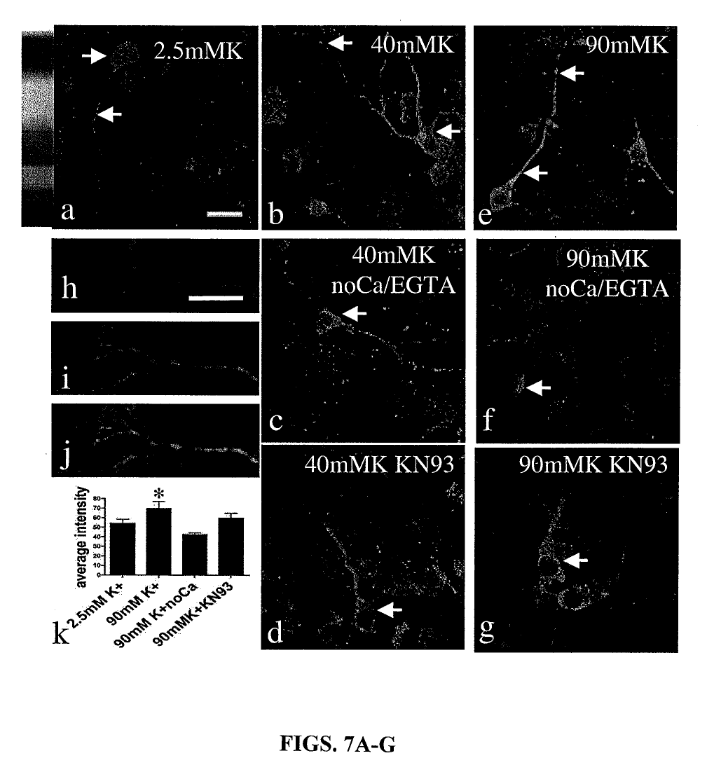

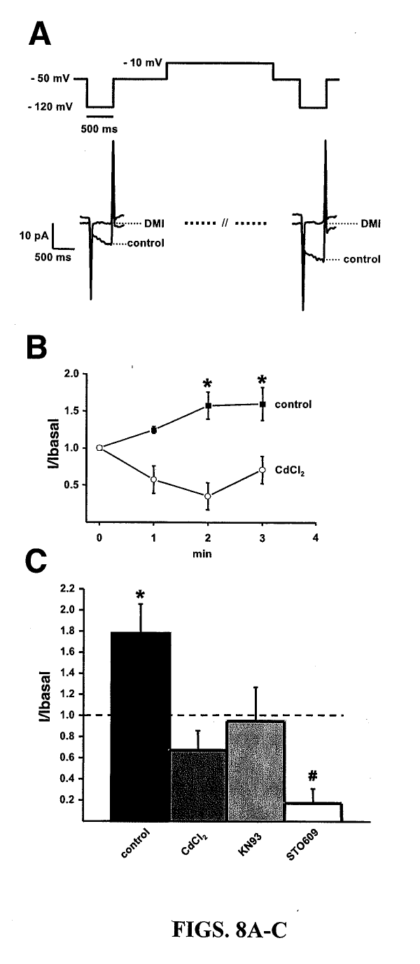

The inventors sought to extend findings of Ca²⁺-sensitive modulation of NET trafficking to primary noradrenergic neurons. SCG neurons synthesize and release NE and express NET (Schroeter et al., 2000), and exhibit depolarization augmented surface NET expression as determined with an ectodomain NET antibody (Savchenko et al., 2003). The inventors implemented the latter paradigm to examine the profile of surface NET immunoreactivity in cultured neurons in response to Ca²⁺ manipulations. As shown in FIG. 8A, exposure of the NET43408 ectodomain epitope is low, and largely restricted to the cell soma, in SCG cells cultured in basal medium containing 2.5 mM K⁺ (FIG. 8A). When medium is switched to 40 mM K⁺ for 5 min prior to fixation and staining in the absence of detergents, the inventors observed a significant increase in surface NET labeling with a particular increase in the extent of process labeling (arrow in (FIG. 8B)), which becomes even more evident at 90 mM K⁺ (FIG. 8E). If SCG cells are switched to Ca²⁺-free medium prior to high K⁺ stimulation, elevation of NET surface labeling is not apparent (FIG. 8C, FIG. 8F). Depolarization triggered surface expression was also found to be CaMK dependent as high potassium buffer failed to increase surface NET density in when cultures were preincubated with KN93 (FIGS. 7A-D, G). To quantitate NET surface density, pixel intensities were captured from NET surface epitope-positive neuronal processes (FIG. 7H) that co-labeled with anti-tubulin (FIG. 7I, FIG. 7J). A significant increase in surface NET-labeling was detected following shift of cells to 90 mM K⁺. This stimulation was lost in cells either cultured in the absence of Ca²⁺ or when coincubated with KN93 during high K+ stimulation in normal Ca²⁺ medium (FIG. 7K).

### Example 9

### Electrical Stimulation of Noradrenergic Neurons Induces a CaMKI and CaMKII Dependent Elevation in NET Currents

In NE transport studies, [³H]NE uptake was also augmented in SCG cultures by elevated K⁺ in a Ca²⁺-dependent manner. To achieve a more physiological paradigm and to bring these findings to the single neuron level, NET-dependent currents (Galli et al., 1995) were monitored in patch clamped SCG neurons, using a voltage protocol to trigger Ca²⁺ influx in normal medium via voltage-sensitive Ca²⁺ channels (Binda et al., 2006). Individual neurons were clamped at −50 mV and NET currents were elicited in a 500 msec step to −120 mV, with NET currents defined by antidepressant (DMI) subtraction as described in Methods. As shown in FIG. 8A, this step generates DMI-sensitive inward transient and leak currents. When neurons are depolarized (2 sec) to −10 mV to elicit Ca²⁺ entry prior to the −120 mV test pulse, the inventors recorded a time-dependent increase in DMI-sensitive currents (FIG. 8A, B) that reached ˜180% of control levels. Inclusion of the inorganic Ca²⁺ channel blocker CdCl₂ in the bath prevented the increase in NET current triggered by prior depolarization (FIG. 8B, C). Additionally, coincubations with either KN93 or STO-609 during the test pulse in normal Ca²⁺ medium also prevented the stimulation-elicited increase in NET currents. STO609 actually converted the stimulation-induced increase into a stimulation-elicited decrease in NET currents, suggesting that CaMKI may offset reductions in NET surface expression that may be triggered by other depolarization-elicited signaling pathways.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,554,101
U.S. Pat. No. 4,659,774
U.S. Pat. No. 4,682,195
U.S. Pat. No. 4,683,202
U.S. Pat. No. 4,683,202
U.S. Pat. No. 4,684,611
U.S. Pat. No. 4,816,571
U.S. Pat. No. 4,879,236
U.S. Pat. No. 4,952,500
U.S. Pat. No. 4,959,463
U.S. Pat. No. 5,141,813
U.S. Pat. No. 5,214,136
U.S. Pat. No. 5,223,618
U.S. Pat. No. 5,264,566
U.S. Pat. No. 5,302,523
U.S. Pat. No. 5,322,783
U.S. Pat. No. 5,378,825
U.S. Pat. No. 5,384,253
U.S. Pat. No. 5,384,253
U.S. Pat. No. 5,384,253
U.S. Pat. No. 5,428,148
U.S. Pat. No. 5,446,137
U.S. Pat. No. 5,464,765
U.S. Pat. No. 5,466,786
U.S. Pat. No. 5,470,967
U.S. Pat. No. 5,538,877
U.S. Pat. No. 5,538,880
U.S. Pat. No. 5,539,082
U.S. Pat. No. 5,539,082
U.S. Pat. No. 5,550,318
U.S. Pat. No. 5,554,744
U.S. Pat. No. 5,563,055
U.S. Pat. No. 5,563,055
U.S. Pat. No. 5,574,146
U.S. Pat. No. 5,580,859
U.S. Pat. No. 5,589,466
U.S. Pat. No. 5,591,616
U.S. Pat. No. 5,602,240
U.S. Pat. No. 5,602,244
U.S. Pat. No. 5,610,042
U.S. Pat. No. 5,610,289
U.S. Pat. No. 5,614,617
U.S. Pat. No. 5,623,070
U.S. Pat. No. 5,645,897
U.S. Pat. No. 5,652,099
U.S. Pat. No. 5,656,610
U.S. Pat. No. 5,670,663
U.S. Pat. No. 5,672,697
U.S. Pat. No. 5,681,947
U.S. Pat. No. 5,700,922
U.S. Pat. No. 5,702,932
U.S. Pat. No. 5,705,629
U.S. Pat. No. 5,708,154
U.S. Pat. No. 5,714,331
U.S. Pat. No. 5,714,331
U.S. Pat. No. 5,714,606
U.S. Pat. No. 5,719,262
U.S. Pat. No. 5,719,262
U.S. Pat. No. 5,736,336
U.S. Pat. No. 5,736,336
U.S. Pat. No. 5,736,524
U.S. Pat. No. 5,763,167
U.S. Pat. No. 5,766,855
U.S. Pat. No. 5,766,855
U.S. Pat. No. 5,773,571
U.S. Pat. No. 5,777,092
U.S. Pat. No. 5,780,448
U.S. Pat. No. 5,786,461
U.S. Pat. No. 5,786,461
U.S. Pat. No. 5,789,215
U.S. Pat. No. 5,792,847
U.S. Pat. No. 5,858,988
U.S. Pat. No. 5,859,221
U.S. Pat. No. 5,871,986
U.S. Pat. No. 5,872,232
U.S. Pat. No. 5,886,165
U.S. Pat. No. 5,891,625
U.S. Pat. No. 5,891,625
U.S. Pat. No. 5,908,845
U.S. Pat. No. 5,925,565
U.S. Pat. No. 5,928,906
U.S. Pat. No. 5,929,237
U.S. Pat. No. 5,935,819
U.S. Pat. No. 5,945,100
U.S. Pat. No. 5,981,274
U.S. Pat. No. 5,994,624
U.S. Pat. No. 4,554,101
U.S. Publn. 20040115703
Aletta et al., J. Biol. Chem., 271:20930-20934, 1996.
Almendro et al., J. Immunol., 157(12):5411-21, 1996.
Angel et al., Cell, 49:729, 1987b.
Angel et al., Mol. Cell. Biol., 7:2256, 1987a.
Apparsundaram et al., J. Pharmacol. Exp. Ther., 287:733-743, 1998.
Apparsundaram et al., J. Pharmacol. Exp. Ther., 299:666-677, 2001.
Atchison and Perry, Cell, 46:253, 1986.
Atchison and Perry, Cell, 48:121, 1987.
Ausubel et al., In: Current Protocols in Molecular Biology, John, Wiley & Sons, Inc, NY, 1994.
Baichwal and Sugden, In: Gene Transfer, Kucherlapati (Ed.), Plenum Press, NY, 117-148, 1986.
Banerji et al., Cell, 27:299, 1981.
Banerji et al., Cell, 35:729, 1983.
Bates, Mol. Biotechnol., 2(2):135-145, 1994.
Battraw and Hall, Theor. App. Genet., 82(2):161-168, 1991.
Berkhout et al., Cell, 59:273, 1989.
Berridge and Waterhouse, Brain Res. Brain Res. Rev., 42:33-84, 2003.
Berridge, Neuron., 21:13-26, 1998.
Bhattacharjee et al., J. Plant Bioch. Biotech., 6(2):69-73. 1997.
Binda et al., Neuropharmacology, 50:354-361, 2006.
Blackman et al., Biophys. J., 71(1):194-208, 1996.
Blakely and DeFelice, Mol. Pharmacol., 71:1206-1208, 2007.
Blakely et al., Physiology, 20:225-231, 2005.
Blanar et al., EMBO J., 8:1139, 1989.
Bodine and Ley, EMBO J., 6:2997, 1987.
Bohm et al., J. Amer. Coll. Cardiol., 25(1):146-153, 1995.
Boshart et al., Cell, 41:521, 1985.
Bosze et al., EMBO J., 5:1615, 1986.
Bouchard et al., Prog. Neurobiol., 69:391-418, 2003.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Carbonelli et al., FEMS Microbiol. Lett., 177(1):75-82, 1999.
Celander and Haseltine, J. Virology, 61:269, 1987.
Celander et al., J. Virology, 62:1314, 1988.
Chandler et al., Cell, 33:489, 1983.
Chandler et al., Proc. Natl. Acad. Sci. USA, 94(8):3596-601, 1997.
Chang et al., Mol. Cell. Biol., 9:2153, 1989.
Chatterjee et al., Proc. Natl. Acad. Sci. USA., 86:9114, 1989.
Chen and Okayama, Mol. Cell. Biol., 7(8):2745-2752, 1987.
Chi et al., Neuron., 38:69-78, 2003.
Choi et al., Cell, 53:519, 1988.
Christou et al., Proc. Natl. Acad. Sci. USA, 84(12):3962-3966, 1987.
Cocea, Biotechniques, 23(5):814-816, 1997.
Cohen et al., J. Cell. Physiol., 5:75, 1987.
Costa et al., Mol. Cell. Biol., 8:81, 1988.
Coupar et al., Gene, 68:1-10, 1988.
Cripe et al., EMBO J., 6:3745, 1987.
Culotta and Hamer, Mol. Cell. Biol., 9:1376, 1989.
D'Halluin et al., Plant Cell, 4(12):1495-1505, 1992.
Dandolo et al., J Virology, 47:55, 1983.
De Villiers et al., Nature, 312:242, 1984.
Deschamps et al., Science, 230:1174, 1985.
Donai et al., Neurosci. Lett., 293:111-114, 2000.
Edbrooke et al., Mol. Cell. Biol., 9:1908, 1989.
Edlund et al., Science, 230:912, 1985.
Egholm et al., Nature, 365(6446):566-568, 1993.
Eisenhofer, Pharmacol. Ther., 91:35-62, 2001.
EP 266,032
EPO 0273085
Esler et al., Hypertension, 3:149-156, 1981.
Fagan et al., J. Biol. Chem., 271:12438-12444, 1996.
Fechheimer, et al., Proc Natl. Acad. Sci. USA, 84:8463-8467, 1987.
Fechheimer, et al., Proc Natl. Acad. Sci. USA, 84:8463-8467, 1987.
Feng and Holland, Nature, 334:6178, 1988.
Firak and Subramanian, Mol. Cell. Biol., 6:3667, 1986.
Foecking and Hofstetter, Gene, 45:101, 1986.
Fraley et al., Proc. Natl. Acad. Sci. USA, 76:3348-3352, 1979.
Fujita et al., Cell, 49:357, 1987.
Gadea et al., J. Neurochem., 80:634-645, 2002.
Gailly, Cell Calcium, 24:293-304, 1998.
Galli et al., J. Exp. Biol., 198:2197-2212, 1995.
Gether et al., Trends Pharmacol. Sci., 27:375-383, 2006. Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Gloss et al., EMBO J., 6:3735, 1987.
Godbout et al., Mol. Cell. Biol., 8:1169, 1988.
Goodboum and Maniatis, Proc. Natl. Acad. Sci. USA, 85:1447, 1988.
Goodboum et al., Cell, 45:601, 1986.
Gopal, Mol. Cell. Biol., 5:1188-1190, 1985.
Gorman and Kent, J. Clin. Psychiatry, 60(4):33-38, 1999. Graham and Van Der Eb, Virology, 52:456-467, 1973.
Greene et al., Immunology Today, 10:272, 1989.
Grosschedl and Baltimore, Cell, 41:885, 1985.
Gu et al., J. Biol. Chem., 269:7124-7130, 1994.
Hahn and Blakely, Pharmacogenomics J, 2:217-235, 2002.
Harland and Weintraub, J. Cell Biol., 101(3):1094-1099, 1985.
Haslinger and Karin, Proc. Natl Acad. Sci. USA., 82:8572, 1985.
Hauber and Cullen, J. Virology, 62:673, 1988.
He et al., Plant Cell Reports, 14 (2-3):192-196, 1994.
Hen et al., Nature, 321:249, 1986.
Hensel et al., Lymphokine Res., 8:347, 1989.
Herr and Clarke, Cell, 45:461, 1986.
Hirochika et al., J. Virol., 61:2599, 1987.
Hirsch et al., Mol. Cell. Biol., 10:1959, 1990.
Holbrook et al., Virology, 157:211, 1987.
Hook and Means, Annu. Rev. Pharmacol. Toxicol., 41:471-505, 2001.
Horlick and Benfield, Mol. Cell. Biol., 9:2396, 1989.
Hou and Lin, Plant Physiology, 111:166, 1996.
Huang et al., Cell, 27:245, 1981.
Hug et al., Mol. Cell. Biol., 8(8):3065-79, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Inouye and Inouye, Nucleic Acids Res., 13:3101-3109, 1985.
Iversen, Biochem. Pharmacol., 23:1927-1935, 1974.
Jakobovits et al., Mol. Cell. Biol., 8:2555, 1988.
Jameel and Siddiqui, Mol. Cell. Biol., 6:710, 1986.
Jayanthi et al., Brit. J. Pharmacol., 129:465-470, 2000.
Jayanthi et al., J. Biol. Chem., 281(33):23326-40, 2006.
Jaynes et al., Mol. Cell. Biol., 8:62, 1988.
Johnson et al., Mol. Cell. Biol., 9:3393, 1989.
Kadesch and Berg, Mol. Cell. Biol., 6:2593, 1986.
Kaeppler et al., Plant Cell Reports, 9:415-418, 1990.
Kaneda et al., Science, 243:375-378, 1989.
Kantor et al., J. Neurosci., 19:3801-3808, 1999.
Kantor et al., J. Pharmacol. Exp. Ther., 297:1016-1024, 2001.
Kantor et al., J. Pharmacol. Exp. Ther., 311:1044-1051, 2004.
Karin et al., Mol. Cell. Biol., 7:606, 1987.
Katinka et al., Cell, 20:393, 1980.
Katinka et al., Nature, 290:720, 1981.
Kato et al., J. Biol. Chem., 266:3361-3364, 1991.
Kawamoto et al., Mol. Cell. Biol., 8:267, 1988.
Keller et al., Neuroscience, 139(3):931-46, 2006.
Kiledjian et al., Mol. Cell. Biol., 8:145, 1988.
Kippenberger et al., J. Neurochem., 73:1024-1032, 1999.
Klamut et al., Mol. Cell. Biol., 10: 193, 1990.
Klimek et al., J. Neurosci., 17(21):8451-8458, 1997.
Koch et al., Mol. Cell. Biol., 9:303, 1989.
Kornberg and Baker, DNA Replication, 2nd Ed., Freeman, San Francisco, 1992.
Kraus et al., FEBS Lett., 428(3):165-70, 1998.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984a.
Kriegler et al., Cell, 53:45, 1988.
Kriegler et al., In: Cancer Cells 2/Oncogenes and Viral Genes, Van de Woude et al. eds, Cold Spring Harbor, Cold Spring Harbor Laboratory, 1984b.
Kuhl et al., Cell, 50:1057, 1987.
Kunz et al., Nucl. Acids Res., 17:1121, 1989.
Kyte and Doolittle, J. Mol. Biol., 157(1):105-132, 1982.
Lareyre et al., J. Biol. Chem., 274(12):8282-90, 1999.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Lazzeri, Methods Mol. Biol., 49:95-106, 1995.
Lee et al., DNA Cell Biol., 16(11):1267-75, 1997.
Lee et al., Korean J. Genet., 11(2):65-72, 1989.
Lee et al., Mol. Endocrinol., 2: 404-411, 1988.
Lee et al., Nature, 294:228, 1981.
Levenson et al., Hum. Gene Ther., 9(8):1233-1236, 1998.
Levinson et al., Nature, 295:79, 1982.
Lin et al., Mol. Cell. Biol., 10:850, 1990.
Luria et al., EMBO J., 6:3307, 1987.
Lusky and Botchan, Proc. Natl. Acad. Sci. USA., 83:3609, 1986.
Lusky et al., Mol. Cell. Biol., 3:1108, 1983.
Macejak and Sarnow, Nature, 353:90-94, 1991.
Majors and Varmus, Proc. Natl. Acad. Sci. USA., 80:5866, 1983.
Maniatis, et al., Molecular Cloning, A Laboratory Manual, Cold Spring Harbor Press, Cold Spring Harbor, N.Y., 1988.
McNeall et al., Gene, 76:81, 1989.
Miksicek et al., Cell, 46:203, 1986.
Miner et al., J. Neurosci., 26:1571-1578, 2006.
Mordacq and Linzer, Genes and Dev., 3:760, 1989.
Moreau et al., Nucl. Acids Res., 9:6047, 1981.
Muesing et al., Cell, 48:691, 1987.
Nabel et al., Science, 244(4910):1342-1344, 1989.
Ng et al., Nuc. Acids Res., 17:601, 1989.
Nichols et al., Nature, 343:647-651, 1990.
Nicolau and Sene, Biochim. Biophys. Acta, 721:185-190, 1982.
Nicolau et al., Methods Enzymol., 149:157-176, 1987.
Nomoto et al., Gene, 236(2):259-71, 1999.
Omirulleh et al., Plant Mol. Biol., 21(3):415-428, 1993.
Ondek et al., EMBO J., 6:1017, 1987.
Ornitz et al., Mol. Cell. Biol., 7:3466, 1987.
Pacholczyk et al., Nature, 350:350-354, 1991.
Palmiter et al., Nature, 300:611, 1982.
PCT Appln. WO 84/03564
PCT Appln. WO 92/20702
PCT Appln. WO 92/17598
PCT Appln. WO 94/09699
PCT Appln. WO 95/06128
Pech et al., Mol. Cell. Biol., 9:396, 1989.
Pelletier and Sonenberg, Nature, 334(6180):320-325, 1988.
Perales et al., Proc. Natl. Acad. Sci. USA, 91:4086-4090, 1994.
Perez-Stable and Constantini, Mol. Cell. Biol., 10:1116, 1990.
Picard and Schaffner, Nature, 307:83, 1984.
Picciotto et al., J. Biol. Chem., 268:26512-26521, 1993.
Pinkert et al., Genes and Dev., 1:268, 1987.
Pliszka, Biol. Psychiatry, 57:1385-1390, 2005.
Ponta et al., Proc. Natl. Acad. Sci. USA., 82:1020, 1985.
Porton et al., Mol. Cell. Biol., 10:1076, 1990.
Potrykus et al., Mol. Gen. Genet., 199(2):169-177, 1985.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Resendez Jr. et al., Mol. Cell. Biol., 8:4579, 1988.
Rhodes et al., Methods Mol. Biol., 55:121-131, 1995.
Ridgeway, In: Vectors. A Survey of Molecular Cloning Vectors and Their Uses, Rodriguez et al. (Eds.), Stoneham: Butterworth, 467-492, 1988.
Rina et al., Brain Res., 911:1-11, 2001.
Ripe et al., Mol. Cell. Biol., 9:2224, 1989.
Rippe, et al., Mol. Cell. Biol., 10:689-695, 1990.
Rittling et al., Nucl. Acids Res., 17:1619, 1989.
Rosen et al., Cell, 41:813, 1988.
Sambrook et al., In: Molecular cloning: a laboratory manual, 2^{nd} Ed., Cold Spring Harbor Laboratory Press, Cold Spring Harbor, N.Y., 1989.
Satake et al., J. Virology, 62:970, 1988.
Savchenko et al., Mol. Cell. Neurosci., 24:1131-1150, 2003.
Schaffner et al., J. Mol. Biol., 201:81, 1988.
Scheit, In: Synthesis and Biological Function, Wiley-Interscience, New York, 171-172, 1980.
Schildkraut, Am. J. Psychiatry, 122:509-522, 1965.
Schmitt et al., J. Neurosci., 25:1281-1290, 2005.
Schroeter et al., J. Comp. Neurol., 420:211-232, 2000.
Schweitzer et al., J Cell Sci., 108(Pt 7):2619-2628, 1995.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stuart et al., Nature, 317:828, 1985.
Sullivan and Peterlin, Mol. Cell. Biol., 7:3315, 1987.
Sumners and Raizada, Am. J. Physiol., 250:C236-244, 1986.
Sung et al., Biochem. Biophys. Res. Commun., 333:671-678, 2005.
Sung et al., J. Neurosci., 23:1697-1709, 2003.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Tokuda et al., Brain Res., 755, 162-166, 1997.
Tokumitsu et al., J. Biol. Chem., 277:15813-15818, 2002.
Treisman, Cell, 42:889, 1985.
Tronche et al., Mol. Biol. Med., 7:173, 1990.
Tronche et al., Mol. Cell. Biol., 9:4759, 1989.
Trudel and Constantini, Genes and Dev., 6:954, 1987.
Tsukada et al., Plant Cell Physiol., 30(4)599-604, 1989.
Tsumaki et al., J. Biol. Chem., 273(36):22861-4, 1998.
Tur-Kaspa et al., Mol. Cell. Biol., 6:716-718, 1986.
Tyndall et al., Nuc. Acids. Res., 9:6231, 1981.
Uchida et al., Brain Res., 809:155-164, 1998.
Uchikawa et al., J. Neurochem., 65:2065-2071, 1995.
Uezu et al., J. Neurochem., 82:585-593, 2002.
Vannice and Levinson, J. Virology, 62:1305, 1988.
Vasseur et al., Proc. Natl. Acad. Sci. USA, 77:1068, 1980.
Verkhratsky, Physiol. Rev., 85:201-279, 2005.
Wagner et al., Proc. Natl. Acad. Sci. USA 87(9):3410-3414, 1990.
Wang and Calame, Cell, 47:241, 1986.
Wang and Oxford, J. Neurophysiol., 84:2888-2895, 2000.
Wayman et al., J. Neurosci., 24:3786-3794, 2004.
Wayman et al., Neuron., 50:897-909, 2006.
Weber et al., Cell, 36:983, 1984.
Weinberger et al. Mol. Cell. Biol., 8:988, 1984.
Weisshoff et al., Eur. J. Biochem., 259:776-788, 1999.
Westphal et al., Science, 280:1258-1261, 1998.
Wilson et al., Science, 244:1344-1346, 1989.
Winoto and Baltimore, Cell, 59:649, 1989.
Wong et al., Gene, 10:87-94, 1980.
Wong et al., Proc. Natl. Acad. Sci. USA, 97:325-330.
Wu and Wu, Adv. Drug Delivery Rev., 12:159-167, 1993.
Wu and Wu, Biochemistry, 27: 887-892, 1988.
Wu and Wu, J. Biol. Chem., 262:4429-4432, 1987.
Wu et al., Biochem. Biophys. Res. Commun., 233(1):221-6, 1997.
Xia and Storm, Nat. Rev. Neurosci., 6:267-276, 2005.
Xu et al., Nat. Neurosci., 3:465-471, 2000.
Yura et al., Brain Res., 738:96-102, 1996.
Yutzey et al. Mol. Cell. Biol., 9:1397, 1989.
Zhao-Emonet et al., Biochim. Biophys. Acta, 1442(2-3):109-19, 1998.
Zhou et al., Nature, 361(6412):543-547, 1993.
Zhu et al., J. Biol. Chem., 280:15649-15658, 2005.

1. An isolated nucleic acid sequence encoding a norepinepherine transporter, wherein the norepinepherine transporter comprises a point mutation at or a deletion of the threonine at position 30 (T30) of the norepinepherine transporter.

2. The isolated nucleic acid of claim 1, wherein the nucleic acid comprises a point mutation at T30.

3. The isolated nucleic acid of claim 2, wherein the mutation is T30A.

4. The isolated nucleic acid of claim 2, wherein the mutation is T30E.

5. The isolated nucleic acid sequence of claim 2, further defined as comprising SEQ ID NO:1 or SEQ ID NO:2.

6. The isolated nucleic acid of claim 2, wherein the mutation is T30G, T30V, 130L, T301, T30P, or T30D.

7. The isolated nucleic acid of claim 2, wherein the mutation is T30F, T30Y, T30W, T30K, T30R, T30H, T30S, T30C, T30M, T30N, T30Q.

8. The isolated nucleic acid of claim 1, wherein the nucleic acid comprises a deletion of T30 of the norepinepherine transporter encoded by the nucleic acid.

9. The isolated nucleic acid of claim 8, wherein the nucleic acid comprises a deletion of amino acids 29-47 of the norepinepherine transporter.

10. The isolated nucleic acid sequence of claim 8, further defined as comprising SEQ ID NO:4.

11. A host cell containing a nucleic acid sequence according to claim 1.

12-16. (canceled)

17. A vector comprising the isolated nucleic acid sequence according to claim 1.

18-21. (canceled)

22. A transgenic non-human animal, wherein the transgenic animal expresses a norepinepherine transporter comprising a point mutation at or a deletion of position T30 of the norepinepherine transporter.

23-31. (canceled)

32. A method of screening a candidate modulator of the norepinephrine transporter (NET) comprising (a) administering said candidate modulator to a transgenic animal of claim 22; and (b) measuring the effect of said candidate modulator on NET trafficking or NET function.

33-41. (canceled)

42. A method of screening for a candidate substance that alters norepinepherine transporter activity or trafficking comprising:
a) providing a cell or cell extract expressing a norepinepherine transporter of claim 1;
b) exposing the cell or cell extract to a candidate substance;
c) measuring binding of the candidate substance to the norepinepherine transporter in step (a);
d) comparing binding of the candidate substance by the norepinepherine transporter of step (a) to binding of the candidate substance by a wild-type norepinepherine transporter,
wherein the ability of the candidate substance to bind to the wild-type norepinepherine transporter, but not the norepinepherine transporter of claim 1, indicates that the candidate substance alters norepinepherine transporter activity trafficking.


SEQUENCE LISTING (13 sequences)

SEQ ID NO: 1 - DNA, 3296 bp, Homo sapiens
atgcttctggcgcggatgaacccgcaggtgcagcccgagaacaacggggcggacacgggtccagagcagccccttcgggcgcgcaaagctgcggagctgctggtggtgaaggagcgcaacggcgtccagtgcctgctggcgccccgcgacggcgacgcgcagccccgggagacctggggcaagaagatcgacttcctgctgtccgtagtcggcttcgcagtggacctggccaacgtgtggcgcttcccctacctctgctacaagaacggcggcggtgccttcttgatcccgtacacactgttccttatcatcgcggggatgcccctgttctacatggagctggctctgggacagtacaaccgggagggggctgccaccgtttggaaaatctgcccattcttcaaaggcgttggctatgctgtcatcctgatcgccctgtacgttggcttctactacaacgtcatcatcgcctggtcactctactacctcttctcctccttcaccctcaacctgccctggaccgactgtggccacacctggaacagccccaactgtaccgaccccaagctcctcaatggctccgtgcttggcaaccacaccaagtactccaagtacaagttcacgccggcagccgagttttatgagcgtggtgtcctgcaccttcacgagagcagcgggattcatgacatcggcctgccccagtggcagctcttgctctgtctgatggtcgtcgtcatcgtcttgtattttagcctctggaaaggggtgaagacatcaggaaaggtggtgtggatcacagccacgctgccttacttcgtgctgttcgtgctcctggtccatggcgtcacgctgcccggagcctccaatggcatcaatgcctacctgcacatcgacttctaccgcttgaaagaggccacggtatggattgatgccgcaactcagatatttttttccttgggggctggatttggagtattgattgcatttgccagttacaacaaatttgacaacaactgttacagggatgccctgctgaccagcagcatcaactgtatcaccagcttcgtctctgggttcgccatcttctccatccttggttacatggcccatgaacacaaggtcaacattgaggatgtggccacagaaggagctggcctagtgttcatcctgtatccagaggccatttctaccctgtctggatctacattctgggctgttgtgtttttcgtcatgctcctggcgctgggccttgacagctcaatgggaggcatggaggctgtcatcacgggcctggcagatgacttccaggtcctgaagcgacaccggaaactcttcacatttggcgtcaccttcagcactttccttctcgccctgttctgcataaccaagggtggaatttacgtcttgaccctcctggacacctttgctgcgggcacctccatcctttttgctgtcctcatggaagccatcggagtttcctggttttatggagtggacaggttcagcaacgacatccagcagatgatggggttcaggccgggtctatactggagactgtgctggaagttcgtcagtcctgccttcctcctgttcgtggttgtggtcagcatcatcaacttcaagccactcacctacgacgactacatcttcccgccctgggccaactgggtggggtggggcatcgccctgtcctccatggtcctggtgcccatctacgtcatctataagttcctcagcacgcagggctctctttgggagagactggcctatggcatcacgccagagaacgagcaccacctggtggctcagagggacatcagacagttccagttgcaacactggctggccatctgagcctgcctggaggagaaggaggaacccccatgccaatgtccaggtcacaggcatccgctgcgctcccacctcggacaccatcttgggattcctcccctggaagttgtcctttctgatcctctcttcttttcccatttacaaatgatttcgtgactgtagtttttgttcaccttctgtgcatctggcctgggggctgttagctcagaggagaggagcaaacaggaaaatgacttctgttctgtccccgctgttttgggggaagtctctcccactttgggatcctgctgaagctaggttcatgaggtcggaaatccccaccacatttgcctagactttgggcacaggagttcttagtccaccaaatcagagagaggatgggcttttgatcagatacccctcccaaaaaaaaaaaaaactaaaactaaagcaaaaatcaaacaaaatctggctgagtttagtggggtggttggggaaggtacatagaccctcctcttgcccaccctagacagccctctcatgtctgaacctcagcctgggagttagatttatttgtctctaaaatgaagtcagtggatagatgctttgagggattttgagtagaaacattcatagttaattttcactctggccaatctgagtttgatgtgtgtgttctggaacattcctccagcttttggtggtcagatggcccagagatatgggggacaggaggaagagggtaaatgaaccacagtgagcaggttctaggaggtacctgcatcagacaagctggtggaggccacgtggcaagccacatctactgaggcctcatgctgctcttgctctgtaagacacggagcccagaaacccatctgcacttcctgagacctgcctggggaaacgggggcagggaccaagtgaggcctcatgtgtgtcttcaccgtgctgtcctcacaaggccaggtgggtgcccaaagggagcctgacaggctgttgtgttaatttattgttcttgcacacctgcacagcctccctctggggatcccacctggagtggaccaggggtcttgagaaatggagagttggctgcaaaaactctcatgcactagatgtggcaccttggagggcagggtgagacaagcagcccagaaatactctctcaagtggaggggagaattttgagagtggatggaacagtttggtggtttcagagaatttctaggtttctacttggatctacttctgatacaaacttgcacttggtgccctctggtggtgtttagttttagttccgtaagagaaatgattcctagtttgctaaattggtggcatctttgggaggggtttctgtttatggttagagtctcttacacccttgttggagggattcttattctgactgtgggagctcctgttgcgggatcttgggaaaaaataaagaagccgctgcattcgcacgtcaa

SEQ ID NO: 2 - DNA, 3296 bp, Homo sapiens
atgcttctggcgcggatgaacccgcaggtgcagcccgagaacaacggggcggacacgggtccagagcagccccttcgggcgcgcaaagaagcggagctgctggtggtgaaggagcgcaacggcgtccagtgcctgctggcgccccgcgacggcgacgcgcagccccgggagacctggggcaagaagatcgacttcctgctgtccgtagtcggcttcgcagtggacctggccaacgtgtggcgcttcccctacctctgctacaagaacggcggcggtgccttcttgatcccgtacacactgttccttatcatcgcggggatgcccctgttctacatggagctggctctgggacagtacaaccgggagggggctgccaccgtttggaaaatctgcccattcttcaaaggcgttggctatgctgtcatcctgatcgccctgtacgttggcttctactacaacgtcatcatcgcctggtcactctactacctcttctcctccttcaccctcaacctgccctggaccgactgtggccacacctggaacagccccaactgtaccgaccccaagctcctcaatggctccgtgcttggcaaccacaccaagtactccaagtacaagttcacgccggcagccgagttttatgagcgtggtgtcctgcaccttcacgagagcagcgggattcatgacatcggcctgccccagtggcagctcttgctctgtctgatggtcgtcgtcatcgtcttgtattttagcctctggaaaggggtgaagacatcaggaaaggtggtgtggatcacagccacgctgccttacttcgtgctgttcgtgctcctggtccatggcgtcacgctgcccggagcctccaatggcatcaatgcctacctgcacatcgacttctaccgcttgaaagaggccacggtatggattgatgccgcaactcagatatttttttccttgggggctggatttggagtattgattgcatttgccagttacaacaaatttgacaacaactgttacagggatgccctgctgaccagcagcatcaactgtatcaccagcttcgtctctgggttcgccatcttctccatccttggttacatggcccatgaacacaaggtcaacattgaggatgtggccacagaaggagctggcctagtgttcatcctgtatccagaggccatttctaccctgtctggatctacattctgggctgttgtgtttttcgtcatgctcctggcgctgggccttgacagctcaatgggaggcatggaggctgtcatcacgggcctggcagatgacttccaggtcctgaagcgacaccggaaactcttcacatttggcgtcaccttcagcactttccttctcgccctgttctgcataaccaagggtggaatttacgtcttgaccctcctggacacctttgctgcgggcacctccatcctttttgctgtcctcatggaagccatcggagtttcctggttttatggagtggacaggttcagcaacgacatccagcagatgatggggttcaggccgggtctatactggagactgtgctggaagttcgtcagtcctgccttcctcctgttcgtggttgtggtcagcatcatcaacttcaagccactcacctacgacgactacatcttcccgccctgggccaactgggtggggtggggcatcgccctgtcctccatggtcctggtgcccatctacgtcatctataagttcctcagcacgcagggctctctttgggagagactggcctatggcatcacgccagagaacgagcaccacctggtggctcagagggacatcagacagttccagttgcaacactggctggccatctgagcctgcctggaggagaaggaggaacccccatgccaatgtccaggtcacaggcatccgctgcgctcccacctcggacaccatcttgggattcctcccctggaagttgtcctttctgatcctctcttcttttcccatttacaaatgatttcgtgactgtagtttttgttcaccttctgtgcatctggcctgggggctgttagctcagaggagaggagcaaacaggaaaatgacttctgttctgtccccgctgttttgggggaagtctctcccactttgggatcctgctgaagctaggttcatgaggtcggaaatccccaccacatttgcctagactttgggcacaggagttcttagtccaccaaatcagagagaggatgggcttttgatcagatacccctcccaaaaaaaaaaaaaactaaaactaaagcaaaaatcaaacaaaatctggctgagtttagtggggtggttggggaaggtacatagaccctcctcttgcccaccctagacagccctctcatgtctgaacctcagcctgggagttagatttatttgtctctaaaatgaagtcagtggatagatgctttgagggattttgagtagaaacattcatagttaattttcactctggccaatctgagtttgatgtgtgtgttctggaacattcctccagcttttggtggtcagatggcccagagatatgggggacaggaggaagagggtaaatgaaccacagtgagcaggttctaggaggtacctgcatcagacaagctggtggaggccacgtggcaagccacatctactgaggcctcatgctgctcttgctctgtaagacacggagcccagaaacccatctgcacttcctgagacctgcctggggaaacgggggcagggaccaagtgaggcctcatgtgtgtcttcaccgtgctgtcctcacaaggccaggtgggtgcccaaagggagcctgacaggctgttgtgttaatttattgttcttgcacacctgcacagcctccctctggggatcccacctggagtggaccaggggtcttgagaaatggagagttggctgcaaaaactctcatgcactagatgtggcaccttggagggcagggtgagacaagcagcccagaaatactctctcaagtggaggggagaattttgagagtggatggaacagtttggtggtttcagagaatttctaggtttctacttggatctacttctgatacaaacttgcacttggtgccctctggtggtgtttagttttagttccgtaagagaaatgattcctagtttgctaaattggtggcatctttgggaggggtttctgtttatggttagagtctcttacacccttgttggagggattcttattctgactgtgggagctcctgttgcgggatcttgggaaaaaataaagaagccgctgcattcgcacgtcaa

SEQ ID NO: 3 - DNA, 3296 bp, Homo sapiens
atgcttctggcgcggatgaacccgcaggtgcagcccgagaacaacggggcggacacgggtccagagcagccccttcgggcgcgcaaaactgcggagctgctggtggtgaaggagcgcaacggcgtccagtgcctgctggcgccccgcgacggcgacgcgcagccccgggagacctggggcaagaagatcgacttcctgctgtccgtagtcggcttcgcagtggacctggccaacgtgtggcgcttcccctacctctgctacaagaacggcggcggtgccttcttgatcccgtacacactgttccttatcatcgcggggatgcccctgttctacatggagctggctctgggacagtacaaccgggagggggctgccaccgtttggaaaatctgcccattcttcaaaggcgttggctatgctgtcatcctgatcgccctgtacgttggcttctactacaacgtcatcatcgcctggtcactctactacctcttctcctccttcaccctcaacctgccctggaccgactgtggccacacctggaacagccccaactgtaccgaccccaagctcctcaatggctccgtgcttggcaaccacaccaagtactccaagtacaagttcacgccggcagccgagttttatgagcgtggtgtcctgcaccttcacgagagcagcgggattcatgacatcggcctgccccagtggcagctcttgctctgtctgatggtcgtcgtcatcgtcttgtattttagcctctggaaaggggtgaagacatcaggaaaggtggtgtggatcacagccacgctgccttacttcgtgctgttcgtgctcctggtccatggcgtcacgctgcccggagcctccaatggcatcaatgcctacctgcacatcgacttctaccgcttgaaagaggccacggtatggattgatgccgcaactcagatatttttttccttgggggctggatttggagtattgattgcatttgccagttacaacaaatttgacaacaactgttacagggatgccctgctgaccagcagcatcaactgtatcaccagcttcgtctctgggttcgccatcttctccatccttggttacatggcccatgaacacaaggtcaacattgaggatgtggccacagaaggagctggcctagtgttcatcctgtatccagaggccatttctaccctgtctggatctacattctgggctgttgtgtttttcgtcatgctcctggcgctgggccttgacagctcaatgggaggcatggaggctgtcatcacgggcctggcagatgacttccaggtcctgaagcgacaccggaaactcttcacatttggcgtcaccttcagcactttccttctcgccctgttctgcataaccaagggtggaatttacgtcttgaccctcctggacacctttgctgcgggcacctccatcctttttgctgtcctcatggaagccatcggagtttcctggttttatggagtggacaggttcagcaacgacatccagcagatgatggggttcaggccgggtctatactggagactgtgctggaagttcgtcagtcctgccttcctcctgttcgtggttgtggtcagcatcatcaacttcaagccactcacctacgacgactacatcttcccgccctgggccaactgggtggggtggggcatcgccctgtcctccatggtcctggtgcccatctacgtcatctataagttcctcagcacgcagggctctctttgggagagactggcctatggcatcacgccagagaacgagcaccacctggtggctcagagggacatcagacagttccagttgcaacactggctggccatctgagcctgcctggaggagaaggaggaacccccatgccaatgtccaggtcacaggcatccgctgcgctcccacctcggacaccatcttgggattcctcccctggaagttgtcctttctgatcctctcttcttttcccatttacaaatgatttcgtgactgtagtttttgttcaccttctgtgcatctggcctgggggctgttagctcagaggagaggagcaaacaggaaaatgacttctgttctgtccccgctgttttgggggaagtctctcccactttgggatcctgctgaagctaggttcatgaggtcggaaatccccaccacatttgcctagactttgggcacaggagttcttagtccaccaaatcagagagaggatgggcttttgatcagatacccctcccaaaaaaaaaaaaaactaaaactaaagcaaaaatcaaacaaaatctggctgagtttagtggggtggttggggaaggtacatagaccctcctcttgcccaccctagacagccctctcatgtctgaacctcagcctgggagttagatttatttgtctctaaaatgaagtcagtggatagatgctttgagggattttgagtagaaacattcatagttaattttcactctggccaatctgagtttgatgtgtgtgttctggaacattcctccagcttttggtggtcagatggcccagagatatgggggacaggaggaagagggtaaatgaaccacagtgagcaggttctaggaggtacctgcatcagacaagctggtggaggccacgtggcaagccacatctactgaggcctcatgctgctcttgctctgtaagacacggagcccagaaacccatctgcacttcctgagacctgcctggggaaacgggggcagggaccaagtgaggcctcatgtgtgtcttcaccgtgctgtcctcacaaggccaggtgggtgcccaaagggagcctgacaggctgttgtgttaatttattgttcttgcacacctgcacagcctccctctggggatcccacctggagtggaccaggggtcttgagaaatggagagttggctgcaaaaactctcatgcactagatgtggcaccttggagggcagggtgagacaagcagcccagaaatactctctcaagtggaggggagaattttgagagtggatggaacagtttggtggtttcagagaatttctaggtttctacttggatctacttctgatacaaacttgcacttggtgccctctggtggtgtttagttttagttccgtaagagaaatgattcctagtttgctaaattggtggcatctttgggaggggtttctgtttatggttagagtctcttacacccttgttggagggattcttattctgactgtgggagctcctgttgcgggatcttgggaaaaaataaagaagccgctgcattcgcacgtcaa

SEQ ID NO: 4 - DNA, 3236 bp, Homo sapiens
atgcttctggcgcggatgaacccgcaggtgcagcccgagaacaacggggcggacacgggtccagagcagccccttcgagcgccccgcgacggcgacgcgcagccccgggagacctggggcaagaagatcgacttcctgctgtccgtagtcggcttcgcagtggacctggccaacgtgtggcgcttcccctacctctgctacaagaacggcggcggtgccttcttgatcccgtacacactgttccttatcatcgcggggatgcccctgttctacatggagctggctctgggacagtacaaccgggagggggctgccaccgtttggaaaatctgcccattcttcaaaggcgttggctatgctgtcatcctgatcgccctgtacgttggcttctactacaacgtcatcatcgcctggtcactctactacctcttctcctccttcaccctcaacctgccctggaccgactgtggccacacctggaacagccccaactgtaccgaccccaagctcctcaatggctccgtgcttggcaaccacaccaagtactccaagtacaagttcacgccggcagccgagttttatgagcgtggtgtcctgcaccttcacgagagcagcgggattcatgacatcggcctgccccagtggcagctcttgctctgtctgatggtcgtcgtcatcgtcttgtattttagcctctggaaaggggtgaagacatcaggaaaggtggtgtggatcacagccacgctgccttacttcgtgctgttcgtgctcctggtccatggcgtcacgctgcccggagcctccaatggcatcaatgcctacctgcacatcgacttctaccgcttgaaagaggccacggtatggattgatgccgcaactcagatatttttttccttgggggctggatttggagtattgattgcatttgccagttacaacaaatttgacaacaactgttacagggatgccctgctgaccagcagcatcaactgtatcaccagcttcgtctctgggttcgccatcttctccatccttggttacatggcccatgaacacaaggtcaacattgaggatgtggccacagaaggagctggcctagtgttcatcctgtatccagaggccatttctaccctgtctggatctacattctgggctgttgtgtttttcgtcatgctcctggcgctgggccttgacagctcaatgggaggcatggaggctgtcatcacgggcctggcagatgacttccaggtcctgaagcgacaccggaaactcttcacatttggcgtcaccttcagcactttccttctcgccctgttctgcataaccaagggtggaatttacgtcttgaccctcctggacacctttgctgcgggcacctccatcctttttgctgtcctcatggaagccatcggagtttcctggttttatggagtggacaggttcagcaacgacatccagcagatgatggggttcaggccgggtctatactggagactgtgctggaagttcgtcagtcctgccttcctcctgttcgtggttgtggtcagcatcatcaacttcaagccactcacctacgacgactacatcttcccgccctgggccaactgggtggggtggggcatcgccctgtcctccatggtcctggtgcccatctacgtcatctataagttcctcagcacgcagggctctctttgggagagactggcctatggcatcacgccagagaacgagcaccacctggtggctcagagggacatcagacagttccagttgcaacactggctggccatctgagcctgcctggaggagaaggaggaacccccatgccaatgtccaggtcacaggcatccgctgcgctcccacctcggacaccatcttgggattcctcccctggaagttgtcctttctgatcctctcttcttttcccatttacaaatgatttcgtgactgtagtttttgttcaccttctgtgcatctggcctgggggctgttagctcagaggagaggagcaaacaggaaaatgacttctgttctgtccccgctgttttgggggaagtctctcccactttgggatcctgctgaagctaggttcatgaggtcggaaatccccaccacatttgcctagactttgggcacaggagttcttagtccaccaaatcagagagaggatgggcttttgatcagatacccctcccaaaaaaaaaaaaaactaaaactaaagcaaaaatcaaacaaaatctggctgagtttagtggggtggttggggaaggtacatagaccctcctcttgcccaccctagacagccctctcatgtctgaacctcagcctgggagttagatttatttgtctctaaaatgaagtcagtggatagatgctttgagggattttgagtagaaacattcatagttaattttcactctggccaatctgagtttgatgtgtgtgttctggaacattcctccagcttttggtggtcagatggcccagagatatgggggacaggaggaagagggtaaatgaaccacagtgagcaggttctaggaggtacctgcatcagacaagctggtggaggccacgtggcaagccacatctactgaggcctcatgctgctcttgctctgtaagacacggagcccagaaacccatctgcacttcctgagacctgcctggggaaacgggggcagggaccaagtgaggcctcatgtgtgtcttcaccgtgctgtcctcacaaggccaggtgggtgcccaaagggagcctgacaggctgttgtgttaatttattgttcttgcacacctgcacagcctccctctggggatcccacctggagtggaccaggggtcttgagaaatggagagttggctgcaaaaactctcatgcactagatgtggcaccttggagggcagggtgagacaagcagcccagaaatactctctcaagtggaggggagaattttgagagtggatggaacagtttggtggtttcagagaatttctaggtttctacttggatctacttctgatacaaacttgcacttggtgccctctggtggtgtttagttttagttccgtaagagaaatgattcctagtttgctaaattggtggcatctttgggaggggtttctgtttatggttagagtctcttacacccttgttggagggattcttattctgactgtgggagctcctgttgcgggatcttgggaaaaaataaagaagccgctgcattcgcacgtcaa

SEQ ID NO: 5 - protein, 20 aa, Artificial Sequence
  Synthetic peptide
FTCEQALQHPWIAGDTALDK

SEQ ID NO: 6 - protein, 11 aa, Artificial Sequence
  Synthetic peptide
NIHQSVSEQIK

SEQ ID NO: 7 - protein, 10 aa, Artificial Sequence
  Synthetic peptide
YLHDLGIVHR

SEQ ID NO: 8 - protein, 25 aa, Artificial Sequence
  Synthetic peptide
ICDPGLTAFEPEALGNLVEGMDFHR

SEQ ID NO: 9 - protein, 17 aa, Artificial Sequence
  Synthetic peptide
EAFSLFDKDGDGTITTK

SEQ ID NO: 10 - protein, 22 aa, Artificial Sequence
  Synthetic peptide
EADIDGDGQVNYEEFVQMMTAK

SEQ ID NO: 11 - protein, 15 aa, Artificial Sequence
  Synthetic peptide
SLGQNPTEAELQDMI

SEQ ID NO: 12 - protein, 19 aa, Artificial Sequence
  Synthetic peptide
NEVDADGNGTIDFPEFLTM

SEQ ID NO: 13 - protein, 12 aa, Artificial Sequence
  Synthetic peptide
DGNGYISAAELR